(19) United States
(12) Patent Application Publication
(10) Pub. No.: US 2026/0241056 A1
(43) Pub. Date: Aug. 20, 2026

(54) METHODS FOR DISINFECTING ENZYMATICALLY ACTIVE COMPOSITE SYSTEM AND USES THEREOF IN THE FOOD INDUSTRY

(71) Applicant: BETTER JUICE LTD., Ness Ziona (IL)

(72) Inventors: Gali YAROM, Givataim (IL); Eran BLACHINSKY, Kibbutz Harel (IL); Arad DAVID, Ariel (IL)

(73) Assignee: BETTER JUICE LTD., Ness Ziona (IL)

(21) Appl. No.: 19/517,587

(22) PCT Filed: May 30, 2024
(86) PCT No.: PCT/IL2024/050535

§ 371 Date: Mar. 9, 2026

Related U.S. Application Data
(60) Provisional application No. 63/505,435, filed on Jun. 1, 2023.

(51) Int. Cl. A61L 2/18; A23L 2/84; C12N 9/04; C12N 9/10

(52) U.S. Cl. CPC A61L 2/18; A23L 2/84; C12N 9/0006; C12N 9/1051

(57) ABSTRACT

The present invention generally relates to methods for in situ disinfecting enzymatically active composite system comprising enzymatically actives agent, such as proteins or microbial cells, immobilized to a support. In addition, there is provided a method, which employs the enzymatically active composite system for reducing the sugar content in fruit or vegetable juices.

### TECHNICAL FIELD

The present invention generally relates to methods for in situ disinfecting solid resins bonded to enzymatically active agents, such as proteins or microbial cells, in a large scale bioreactor assemblies. This is exemplified in a method for reducing the sugar content of beverages, which employs the in situ disinfection.

### BACKGROUND

In the food industry, antimicrobial disinfectants are routinely used to sanitize and disinfect product contact surfaces. Quaternary ammonium compounds (QACs/QATs) are active antimicrobial substances, approved sanitizers for use on food contact surfaces.

EP 1233057 discloses microbial cells sterilized by adding a cationic or an ampholytic surfactant and a method for producing the same, in process for sterilizing viable microbial cells having produced therein an industrially useful enzyme or the like without deactivating the enzyme or the like. The sterilization treatment is applied to a microbial cell contained in a microbial catalyst.

Application of enzymes in different industries and particularly in the food industry is well known. However, enzymes, being proteins, are highly sensitive to various denaturing conditions (e.g., temperature, pH), particularly when employed as catalysts in solutions. Moreover, enzymes in aqueous homogeneous catalytic systems typically lead to product contamination and are not easily recovered for reuse (Abolpour Homaei et al., J Chem Biol, 2013, 6:185-205).

To overcome these limitations enzymes are immobilized and fixed to- or within solid supports. The solid support systems generally stabilize the structure of the enzymes and, as a consequence, maintain their activities and improve their stability compared to free enzymes in solution. In addition, heterogeneous immobilized enzymes systems allow the easy recovery of both enzymes and products, multiple re-use of enzymes, continuous operation of enzymatic processes, rapid termination of reactions, and greater variety of bioreactor designs. On the other hand, compared with free enzymes, immobilized enzymes commonly show deterioration in activity over time, e.g., due to growth of contaminating microorganism in their vicinity. IA general disadvantage of using isolated enzymes in the food industry is the cost of their isolation and purification, which is many times higher than that of ordinary catalysts (Abolpour Homaei et al., J Chem Biol, 2013, 6:185-205).

Enzymatic processes in the food industry are typically performed in a bioreactor. Secondary contamination and the threat of food borne diseases constitute a major concern when utilizing cells-based bioreactors in the food industry. Thus, disinfection of the bioreactor enzymatic activity is a necessity.

Present processes, which employ solid supports, which comprises enzymatically active agents suffer from several drawbacks, mainly the deterioration of enzymatic activity over time, e.g., due to accumulation of microorganisms over the beads. In particular, such processes which are used in the food and beverage industry are prone and sensitive to such contaminations. While frequent replacement of the supported enzymatically active agent, in order to avoid enzymatic activity deterioration is feasible, it is both cumbersome and wasteful. In addition, in order to prevent contamination (i.e., the food borne diseases) in the bioreactor, such processes require the removal of the enzyme-support system therefrom, which is also burdensome and slows down the production.

Thus, there is an unmet need for an in-situ processes for disinfection and recycling of the solid support and enzymatically active agents, which enable continuous, efficient and clean process of using such solid supports, e.g., in the food industry for prolonged periods.

### SUMMARY

The present invention provides methods for performing enzymatic transformations, while maintaining and reusing immobilized active enzymatic agent(s) (also designated herein enzymatically active composite system). Advantageously, the present methods enable product production within an enzymatic reaction assembly (bioreaction assembly) comprising a bioreactor in a continuously active state for prolonged periods without the need to discard valuable system component(s) and without long breaks in production, which are typically required in order to remove contamination accumulated in the bioreactor.

Advantageously, the present invention enables the in situ disinfection of an enzymatically active composite system while maintaining the mechanical, chemical and/or activity (e.g., enzymatic activity) properties of the composite system.

Remarkably, the method of the present invention is operable for a prolonged period of time, without visible degradation of the enzymatic agent support and/or the appearance of sediments in the liquid flowing through the enzymatic reaction assembly and without the need to exchange batches of enzymatically active composite system, which can be time- and resource consuming.

Specifically, the present method is based on separating the disinfection of the enzymatic bioreaction assembly comprising a bioreactor into two separate steps, each responsible for a disinfection of a separate section of the enzymatic reaction assembly. In particular, according to some embodiments, the present process including disinfection steps (c1) and (c2), which may be performed at any order. Step (c1) involves disinfection of sections of the bioreaction assembly, which are not in direct contact with the bioreactor itself or with the enzymatically active composite system contained therein, thus this step is allowed to employ a more aggressive disinfectant, such as an acid and/or a base. In contrast, step (c2) involves disinfection of the bioreactor interior and sections of the assembly, which are in direct contact therewith or with the enzymatically active composite system contained therein, thus this step requires to employ a more specialized disinfectant, which is compatible with the more sensitive enzymatically active agents and composite systems. The combination of steps (c1) and (c2) advantageously solves the problems set above of the prior methods and allows semi-continuous employment of the bioreactor assembly and production method without unnecessary breaks for replacement of the composite system.

In certain embodiments, a bioreaction assembly used in the method of the present invention is designed to have a bypass circulation section, which, through the use of junctions, enables separation into the section in which the disinfection of step (c1) is carried out and the section in which the disinfection of step (c2) is carried out. This, as detailed herein, advantageously allows the continuous and/or semi-continuous operation of the bioreactor and enzymatically active composite system contained therein to produce the desired product without the need to exchange batches of the enzymatically active composite system.

In certain embodiments, the above bypass set up of the bioreaction assembly enables circulating at least a portion of the starting liquid composition back into a bioreactor during the enzymatic reaction period. Beneficially, this enables to enhance the mixing and monitor the contact duration between a liquid and the enzymatically active composite system, to achieve optimal results.

In certain embodiments, the method of the invention can be used in the food and beverage industry, e.g., for reducing the monosaccharide or disaccharide content of a fruit or vegetable juice. To this end, according to some embodiments, the method may employ the use of food grade chemicals to reduce and possibly even prevent the formation of contaminations within the enzymatic reaction assembly, while maintaining low microbial counts, at an acceptable level in the food and beverage industry.

According to one aspect of the present invention, there is provided a method of performing an enzymatic reaction and disinfecting an enzymatically active composite system, the method comprising: (a) providing an enzymatic reaction assembly, comprising: a liquid feed, which contains a starting liquid composition; a bioreactor, which contains an enzymatically active composite system, the composite system comprising an enzymatically active agent immobilized to a support; a liquid feed outlet line, which extends from the liquid feed to a first junction, wherein the first junction comprises a first port connected to the feed outlet line, a second port connected to a bioreactor inlet line and a third port connected to a circulation line; wherein the bioreactor inlet line is in fluid communication with the bioreactor and the circulation line is in fluid communication with a second junction; a bioreactor outlet line, which extends from the bioreactor to the second junction, wherein the second junction comprises a first port connected to the bioreactor outlet line, a second port connected to the circulation line and a third port connected to a product line; and a product container, which is in fluid communication with the product line; (b) performing an enzymatic reaction, comprising: (b1) flowing the starting liquid composition from the liquid feed through the liquid feed outlet line, the first junction and the bioreactor inlet line into the bioreactor; (b2) contacting the starting liquid composition with the enzymatically active composite system in the bioreactor, thereby inducing an enzymatic reaction, to produce a liquid product composition; (b3) recirculating the liquid product composition from the bioreactor through the bioreactor outlet line, the second junction, the circulation line, the first junction and the bioreactor inlet line, back into the bioreactor, wherein step (b3) is optional and may be performed one or more times; (b4) flowing the liquid product composition from the bioreactor through the bioreactor outlet line, the second junction and the product line into the product container; (c) performing a disinfection, comprising performing steps (c1) and (c2) at any order, wherein step (c1) comprises flowing an acidic solution, flowing a basic solution or flowing both consecutively, through the liquid feed outlet line, the first junction, the circulation line, the second junction and the product line, thereby washing a portion of the assembly, which does not include the bioreactor; and step (c2) comprises contacting the enzymatically active composite system within the bioreactor with an aqueous disinfecting composition, thereby producing a disinfected enzymatically active composite composition dispersed in the aqueous disinfecting composition; and separating the disinfected enzymatically active composite system from the aqueous disinfecting composition, thereby producing a separated disinfected enzymatically active composite system, wherein the aqueous disinfecting composition comprises a quaternary ammonium compound; wherein the enzymatically active composite system is contained within the bioreactor throughout steps (c1) and (c2); wherein the method further comprises repeating one or more times the enzymatic reaction according to (b) after the disinfection of (c).

According to one embodiment of the present invention, the one or more times of the enzymatic reaction according to (b) after the disinfection of (c) comprises a final repeat, wherein the time from an initial performance of the enzymatic reaction according to (b) to the final repeat is at least 30 days, during which the enzymatically active composite system is maintained within the reactor.

According to another embodiment, the aqueous disinfecting composition is a food grade composition.

According to another embodiment, the quaternary ammonium compound is represented by Formula (I):

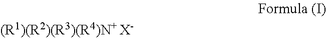

wherein each one of R¹ and R² is independently a methyl; each one of R³ and R⁴ is independently a C₂₋₁₆ alkyl; and X is a halide.

According to another embodiment, X is Cl⁻ and each one of R³ and R⁴ is independently a C₈₋₁₂ alkyl.

According to another embodiment, wherein the quaternary ammonium compound is didecyldimethylammonium chloride.

According to another embodiment, the aqueous disinfecting composition of step (c2) further comprises a potassium salt, a calcium salt, or a combination thereof, at a concentration of at least 0.06M.

According to another embodiment, the aqueous disinfecting composition of step (c2) is an aqueous disinfecting solution.

According to another embodiment, the support is a solid support or a semi-solid support.

According to another embodiment, the enzymatically active composite system comprises the enzymatically active agent bonded to the support, entrapped within the support, encapsulated within the support, embedded in the support or a combination thereof.

According to another embodiment, the enzymatically active composite system is in the form of beads.

According to another embodiment, the support is substantially water insoluble at 25° C. and comprises an anionic polymer comprising a polymeric backbone bonded to a plurality of negatively charged functional groups, selected from the group consisting of: carboxylate, sulfate and sulfonate; and potassium countercations.

According to another embodiment, the potassium cations and the negatively charged functional groups are at a ratio of at least 1:2.

According to another embodiment, the support comprises a combination of alginate and gellan gum or combination of carrageenan and xanthan gum.

According to another embodiment, the enzymatically active agent is isolated enzymes, a plurality of microbial cells or both.

According to another embodiment, the enzymatically active agent comprises a plurality of microbial cells.

According to another embodiment, the enzymatically active agent comprises a plurality of non-proliferating bacterial cells, a plurality of non-proliferating fungal cells or both.

According to another embodiment, the enzymatically active agent catalyzes a transformation of monosaccharides, disaccharides or both into at least one of at least one sugar alcohol; at least one a sugar acid; at least one oligosaccharide; at least one polysaccharide; or any combination thereof.

According to another embodiment, the enzymatically active agent catalyzes a transformation of glucose, fructose, sucrose, or any combination thereof into at least one of sorbitol, gluconic acid, at least one fructooligosaccharides, and any combination thereof.

According to another embodiment, the enzymatically active agent comprises an enzyme selected from the group consisting of cellulose synthase, glucosyltransferase, oxidoreductases, fructosyltransferase, glucose oxidase, glucose isomerase and any combination thereof.

According to another embodiment, the enzyme is glucose fructose oxidase reductase (GFOR), glucose oxidase (GOX) or sucrose fructosyltransferase (SFTase).

According to another embodiment, the enzymatically active agent has a pre-disinfected enzymatic activity in step (b) and a post-disinfected activity upon completion of step (c), wherein the post-disinfected activity is at least 50% of the pre-disinfected enzymatic activity.

According to another embodiment, the enzymatically active composite system of step (b) has an initial microbial count, and wherein step (c) entails disinfecting said enzymatically active composite system, wherein the separated disinfected enzymatically active composite system has a final microbial count; and wherein the initial microbial count is higher than the final microbial count.

According to another embodiment, step (c1) comprises washing the portion of the bioreaction assembly with an aqueous basic solution.

According to another embodiment, the basic solution has pH in the range of 11 to 13.

According to another embodiment, the basic solution comprises NaOH, KOH or both.

According to another embodiment, the NaOH, KOH or both is at a concentration of about 2% w/w.

According to another embodiment, washing the portion of the assembly with an aqueous basic solution is performed at a temperature in the range of 50° C. to 100° C.

According to another embodiment, step (c1) comprises washing the portion of the assembly with an aqueous acidic solution.

According to another embodiment, the acidic solution has pH in the range of 2 to 4.

According to another embodiment, the acidic solution comprises hydrochloric acid, sulfuric acid, phosphoric acid, or nitric acid.

According to another embodiment, the acid is at a concentration of about 1.5% w/w.

According to another embodiment, washing the portion of the assembly with an aqueous acidic solution is performed at a temperature in the range of 50° C. to 100° C.

According to another embodiment, step (c1) comprises (ia) washing the portion of the assembly with the aqueous basic solution, (iia) washing the portion of the assembly with a pH neutral aqueous solution, and (iiia) washing the portion of the assembly with the aqueous acidic solution; or (ib) washing the portion of the assembly with the aqueous acidic solution, (iib) washing the portion of the assembly with a pH neutral aqueous solution, and (iiib) washing the portion of the assembly with the aqueous basic solution.

According to another embodiment, washing the portion of the bioreaction assembly with the pH neutral aqueous solution is performed at a temperature in the range of 50° C. to 100° C.

According to another embodiment, step (c2) comprises maintaining the enzymatically active composite system with the aqueous disinfecting composition in the bioreactor at a temperature in the range of 4° C. to 40° C.

According to another embodiment, reducing the monosaccharide or disaccharide content of a fruit or vegetable juice, wherein the starting liquid composition comprises an initial fruit or vegetable juice, which comprises an initial total monosaccharide and disaccharide concentration; the stating liquid of step (b1) comprises a portion of the initial fruit or vegetable juice; step (b2) comprises contacting the initial fruit or vegetable juice with the enzymatically active composite system in the bioreactor, thereby inducing an enzymatic reaction, which catalyzes a transformation of monosaccharides, disaccharides or both into at least one of at least one sugar alcohol; at least one a sugar acid; at least one oligosaccharide; at least one polysaccharide; or any combination thereof; to produce a processed juice, which has a final total monosaccharide and disaccharide concentration; and wherein the initial total monosaccharide and disaccharide concentration is higher than the final total monosaccharide and disaccharide concentration.

According to another embodiment, the final total monosaccharide and disaccharide concentration is at least 25% lower than the initial total monosaccharide and disaccharide concentration.

According to another embodiment, the initial juice comprises no oligosaccharide and polysaccharide or has an initial total oligosaccharide and polysaccharide concentration and the processed juice has a final total oligosaccharide and polysaccharide concentration, which is higher that the initial total oligosaccharide and polysaccharide concentration.

According to another embodiment, the final total oligosaccharide and polysaccharide concentration is at least 50% higher that the initial total oligosaccharide and polysaccharide concentration.

According to another embodiment, the one or more times of the enzymatic reaction according to (b) after the disinfection of (c) comprises a final repeat, wherein the time from an initial performance of the enzymatic reaction according to (b) to the final repeat is at least 30 days, during which the enzymatically active composite system is maintained within the bioreactor, and wherein a total microbial count of the processed juice produced in step (b) of the final repeat is no more than 1000 CFU/ml.

According to another embodiment, the time from an initial performance of the enzymatic reaction according to (b) to the final repeat is at least 50 days and the total microbial count of the processed juice produced in step (b) of the final repeat is no more than 100 CFU/ml.

According to another embodiment, the time from an initial performance of the enzymatic reaction according to (b) to the final repeat is at least 60 days and the total microbial count of the processed juice produced in step (b) of the final repeat is no more than 100 CFU/ml.

According to another embodiment, the time from an initial performance of the enzymatic reaction according to (b) to the final repeat is at least 70 days and the total microbial count of the processed juice produced in step (b) of the final repeat is no more than 100 CFU/ml.

According to another embodiment, the fruit or vegetable juice is selected from a group consisting of orange juice, lemon juice, grapefruit juice, grape juice, apple juice, pear juice, cherry juice, cranberry juice, mango juice, strawberry juice, pineapple juice, guava juice, peach juice, plum juice, apricot juice, nectarine juice, current juice, raspberry juice, gooseberry juice, blackberry juice, blueberry juice, pomegranate juice, kiwi juice, banana juice, Papaya juice, watermelon juice, cantaloupe juice, coconut juice, passion fruit juice, beetroot juice, tomato juice, celery juice, rhubarb juice and carrot juice.

According to another embodiment, the fruit or vegetable juice is selected from a group consisting of orange juice, grape juice, apple juice, pear juice, cherry juice, strawberry juice, and pineapple juice.

The method for reducing the monosaccharide or disaccharide content of a fruit or vegetable juice, wherein: the aqueous disinfecting composition is a food grade composition; the support comprises a combination of carrageenan and xanthan gum; the enzymatically active agent comprises a plurality of non-proliferating bacterial cells, comprising glucose fructose oxidase reductase (GFOR), glucose oxidase (GOX) or sucrose fructosyltransferase (SFTase); the final total monosaccharide and disaccharide concentration is at least 25% lower than the initial total monosaccharide and disaccharide concentration; the initial juice has no oligosaccharide and polysaccharide concentration or an initial total oligosaccharide and polysaccharide concentration and the processed juice has a final total oligosaccharide and polysaccharide concentration, which is at least 50% higher that the initial total oligosaccharide and polysaccharide concentration; the one or more times of the enzymatic reaction according to (b) after the disinfection of (c) comprises a final repeat, wherein the time from an initial performance of the enzymatic reaction according to (b) to the final repeat is at least 50 days, during which the enzymatically active composite system is maintained within the reactor, and wherein a total microbial count of the processed juice produced in step (b) of the final repeat is no more than 100 CFU/ml; said fruit or vegetable juice is selected from a group consisting of orange juice, grape juice, apple juice, pear juice, cherry juice, strawberry juice, and pineapple juice.

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

### BRIEF DESCRIPTION OF THE FIGURES

FIGS. 1A-1C illustrate block diagrams for a bioreaction assembly 100, according to some embodiments. A general description of the bioreaction assembly 100 is presented in FIG. 1A and further details are provided in FIG. 1B and FIG. 1C.

FIG. 2A illustrate a flowchart of a method of performing an enzymatic reaction and disinfecting an enzymatically active composite system.

FIG. 2B illustrate a flowchart of a method of performing an enzymatic reaction and disinfecting an enzymatically active composite system with optional steps according to the methods of FIGS. 2A and 2B.

### DETAILED DESCRIPTION

Foodborne diseases are illnesses that result from ingestion of contaminated food. Most of foodborne diseases are infections, caused by a variety of bacteria, viruses, and parasites. The sources of the contaminating agents range from being an inherent constituent of the food to inadvertent (or intentional) addition during food production, processing, or preparation.

Disinfection processes adapted to the food industry, result in the removal, killing or deactivation of contaminating agents which are present in food products or on any certain surface of an object, without the risk of harming the food product consumers. However, when applying biological agents immobilized to a solid support having an enzymatic activity in the food industries, disinfection processes and/or disinfection agents may have mal-effect on the immobilized biological agents. The disinfection processes and/or agents may impair the immobilized biological agents' activity and may cause a deterioration of the solid support.

These mal-effects, often require the ceasing of production, the removal of the immobilized biological agents for disinfecting purposes and in many cases also of the replacement of the immobilized biological agents with a new batch. Ceasing production and replacement of immobilized biological agents are time consuming and highly expensive.

The present invention provides a method for performing an enzymatic reaction and disinfecting an enzymatically active composite system, while maintaining the enzymatically active composite system in an active state for prolonged periods, without the need to cease production or to replace the enzymatically active composite system due to disinfection process. The present invention allows the reuse of the enzymatically active composite system following a plurality of operation and disinfection cycles.

Specifically, the deficiencies associated with the ceasing of production requirements of the prior art are overcome by the present method by partition of the disinfection procedure into two separate disinfections procedures applied to different section of the bioreaction assembly, in which the production method is performed. The disinfection steps are divided as portrayed in steps (c2) of disinfection of the bioreactor and enzymatically active composite contained therein; and (c1) of disinfection of the other assembly modules and pipes. This ensures a thorough disinfection of the assembly, without substantial harm to the composite, to achieve a clean-in-place method of the bioreactor assembly and avoid unnecessary maintenance.

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

The singular forms “a”, “an”, and “the” include plural referents unless the context clearly dictates otherwise.

Unless the context clearly requires otherwise, throughout the specification, the words “comprise”, “comprising” and the like are to be construed in an inclusive sense as opposed to an exclusive or exhaustive sense; that is to say, in the sense of “including, but not limited to”.

The word “about”, as used in the specification, should generally be understood to refer to both numbers in a range of numerals, and refers to the numeral ±5%-±10%. Moreover, all numerical ranges herein should be understood to include each whole integer within the range.

According to one aspect, of the present invention provides a method of performing an enzymatic reaction and disinfecting an enzymatically active composite system, the method comprising:
(a) providing an enzymatic reaction assembly, comprising:
   a liquid feed, which contains a starting liquid composition;
   a bioreactor, which contains an enzymatically active composite system, the composite system comprising an enzymatically active agent immobilized to a support;
   a liquid feed outlet line, which extends from the liquid feed to a first junction, wherein the first junction comprises a first port connected to the liquid feed outlet line, a second port connected to a bioreactor inlet line and a third port connected to a circulation line; wherein the bioreactor inlet line is in fluid communication with the bioreactor and the circulation line is in fluid communication with a second junction;
   a bioreactor outlet line, which extends from the bioreactor to the second junction, wherein the second junction comprises a first port connected to the bioreactor outlet line, a second port connected to the circulation line and a third port connected to a product line; and a product container, which is in fluid communication with the product line;
(b) performing an enzymatic reaction, comprising:
   (b1) flowing the starting liquid composition from the liquid feed through the liquid feed outlet line, the first junction and the bioreactor inlet line into the bioreactor;
   (b2) contacting the starting liquid composition with the enzymatically active composite system in the bioreactor, thereby inducing an enzymatic reaction, to produce a liquid product composition;
   (b3) recirculating the liquid product composition from the bioreactor through the bioreactor outlet line, the second junction, the circulation line, the first junction and the bioreactor inlet line, back into the bioreactor, wherein step (b3) is optional and may be performed one or more times;
   (b4) flowing the liquid product composition from the bioreactor through the bioreactor outlet line, the second junction and the product line into the product container;
(c) performing a disinfection, comprising performing steps (c1) and (c2) at any order, wherein
   step (c1) comprises flowing an acidic solution, flowing a basic solution or flowing both consecutively, through the liquid feed outlet line, the first junction, the circulation line, the second junction and the product, thereby
   washing a portion of the assembly, which does not include the bioreactor; and
   step (c2) comprises contacting the enzymatically active composite system within the bioreactor with an aqueous disinfecting composition, thereby producing a disinfected enzymatically active composite composition dispersed in the aqueous disinfecting composition; and separating the disinfected enzymatically active composite system from the aqueous disinfecting composition, thereby producing a separated disinfected enzymatically active composite system, wherein the aqueous disinfecting composition comprises a quaternary ammonium compound;
   wherein the enzymatically active composite system is contained within the bioreactor throughout steps (c1) and (c2);
wherein the method further comprises repeating one or more times the enzymatic reaction according to (b) after the disinfection of (c).

Specific reference is now made to step (a) of the present method. According to some embodiments, the present method includes step (a) of providing an enzymatic reaction assembly.

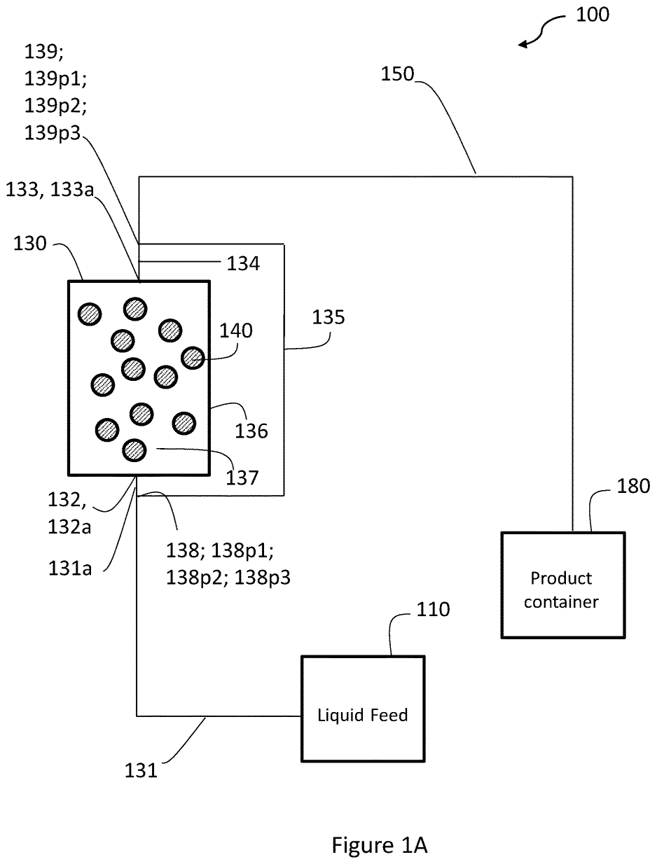

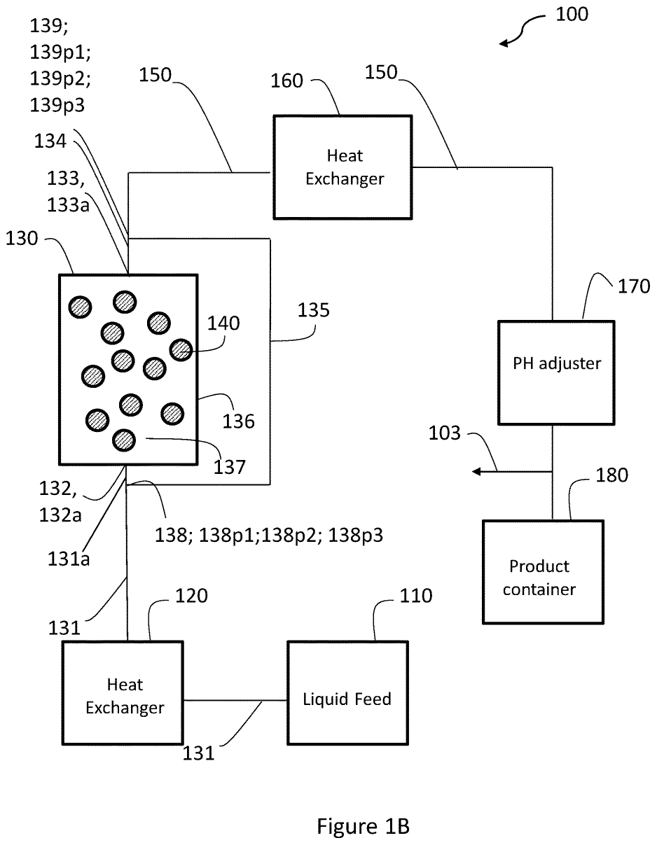

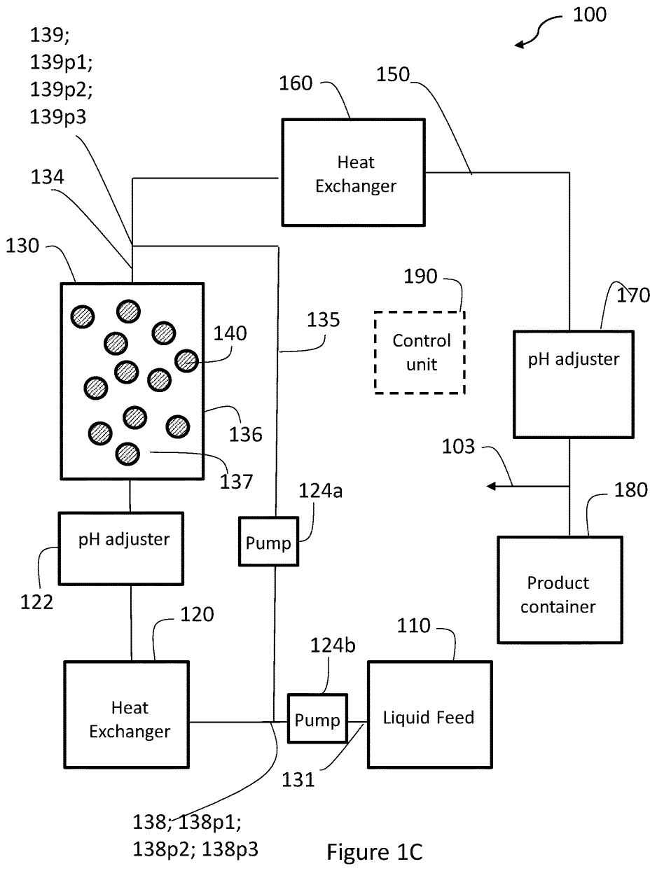

Three optional configurations of enzymatic reaction assembly (bioreaction assembly) 100 are presented in FIG. 1A, FIG. 1B and FIG. 1C, however, any combination of components includes in the enzymatic reaction assembly 100 of FIG. 1A and that of FIG. 1B or FIG. 1C is contemplated.

Reference is now made to FIG. 1A, which illustrates a block diagram schematically representing an enzymatic reaction assembly 100, according to some embodiments.

According to some embodiments, the enzymatic reaction assembly 100 comprises liquid feed 110, connected to a liquid feed outlet line 131, which extends from the liquid feed to a first junction 138.

According to some embodiments, the first junction 138 has at least three inlet and/or outlet ports. According to some embodiments, the first junction 138 has three inlet and/or outlet ports: 138 first port 138p1, second port 138p2 and third port 138p3, according to some embodiments. The first port 138p1 is connected to liquid feed outlet line 131. The second port 138p2 is connected to bioreactor inlet line 131a, according to some embodiments. The third 138p3 port is connected to circulation line 135, according to some embodiments.

According to some embodiments, the first junction 138 has at least three open/close states.

According to some embodiments, in the first open state of the first junction 138, first port 138p1 and second port 138p2 are open and third port 138p3 is closed. Thus, the first open state of the first junction 138 may be employed during step (b1) of the present method. According to some embodiments, in the first open state of the first junction 138 the liquid feed outlet line 131 and bioreactor inlet line 131a are in fluid communication.

According to some embodiments, in the second open state of the first junction 138, the first port 138p1 and the third port 138p3 are open. According to some embodiments, in the second open state of the first junction 138 the second port 138p2 is closed. Thus, the second open state of the first junction 138, wherein the second port 138p2 is closed may be employed during step (c1) of the present method. This, according to some embodiments, allows fluid separation between the bioreactor 130 and the circulation line 135 during step (c1) so that the acidic solution or basic solutions do not contact the enzymatically active composite system within the bioreactor 130.

According to some embodiments, in the second open state of the first junction 138 second port 138p2 may be open. Thus, the second open state of the first junction 138, wherein the second port 138p2 is open may be employed when performing steps (b1), (b2) and (b3) simultaneously. Alternatively, the second open state of the first junction 138, wherein the second port 138p2 is open may be employed when performing steps (b1), (b2), (b3) and (b4) simultaneously. According to some embodiments, in the second open state of the first junction 138 the circulation line 135 and bioreactor inlet line 131a are in fluid communication.

According to some embodiments, in the third open state of the first junction 138, second port 138p2 and third port 138p3 are open and the first port 138p1 is closed. Thus, the third open state of the first junction 138 may be employed when performing steps (b3).

Bioreactor inlet line 131a extends between first junction 138 and bioreactor 130 and is coupled to bioreactor 130 via bioreactor inlet port 132, according to some embodiments. Bioreactor 130 is connected to bioreactor outlet line 134 via bioreactor outlet port 133. Bioreactor outlet line 134 extends between bioreactor 130 and second junction 139, according to some embodiments.

According to some embodiments, the second junction 139 has at least three inlet and/or outlet ports. According to some embodiments, the second junction 139 has three inlet and/or outlet ports: first port 139p1, second port 139p2 and third port 139p3, according to some embodiments. The first port 139p1 is connected to bioreactor outlet line 134. The second port 139p2 is connected to circulation line 135, according to some embodiments. The third 139p3 port is connected to product line 150, according to some embodiments.

According to some embodiments, the second junction 139 has at least three open and/or closed ports. Second junction 139 has three open states, according to some embodiments.

According to some embodiments, in the first open state of the second junction 139, first port 139p1 and third port 139p3 are open and second port 139p2 is closed, according to some embodiments. Thus, the first open state of the second junction 139 may be employed during step (b4) of the present method. According to some embodiments, in the first open state of the second junction 139 the bioreactor outlet line 134 and product line 150 are in fluid communication.

According to some embodiments, in the second open state of the second junction 139, first port 139p1 and second port 139p2 are open. According to some embodiments, in the second open state of the second junction 139, the bioreactor outlet line 134 and circulation line 135 are in fluid communication. According to some embodiments, in the second open state of the second junction 139 the third port 139p3 may be open, closed or alternately opened and closed. This, according to some embodiments, allows control of the product flow into the product container 180 during the recirculation step (b3), as detailed herein. In other words, when the second junction 139 is switched to its second state and the third port 139p3 is open, steps (b3) and (b4) are performed simultaneously. Alternatively, the 139p3 may be closed in the second state, so that steps (b3) may be performed alone. According to some embodiments, in the second open state of the second junction 139 the third port 139p3 is closed. According to some embodiments, in the second open state of the second junction 139 the third port 139p3 is open.

According to some embodiments, in the third open state of the second junction 139 second port 139p2 and third port 139p3 are open. According to some embodiments, in the third open state of the second junction 139 the bioreactor outlet line 134 and circulation line 135 are in fluid communication. According to some embodiments, in the third open state of the second junction 139 first port 139p1 is closed. This, according to some embodiments, allows fluid separation between the bioreactor 130 and the circulation line 135 during step (c1) so that the acidic solution or basic solutions do not contact the enzymatically active composite system within the bioreactor 130.

According to some embodiments, the product line 150 extends between second junction 139 and product tank 180.

As used herein, the term “fluid communication” refers to a path which allows fluid to flow between two components or segments of the assembly of the present invention, wherein said two components can be directly or indirectly joined to each other. Similarly, as used herein, the terms “fluidly coupled” or “fluidly connected” are interchangeable, and refer to a connection between two components that allows fluid to flow from one component to the other, wherein said connection may be direct or indirect via one or more intermediate components enabling fluid flow therethrough.

As used herein, the term “connected” without a qualifier generally means physically connected, or coupled or linked and does not exclude the presence of intermediate elements between the connected elements absent specific contrary language.

According to some embodiments, the various components of assembly 100 are in fluid communication with each other via lines, valves, any other communication appliance known in the art, and combinations thereof. As used herein, the term “line” refers to a fluid supply line, or an element configured to enable fluid communication therethrough, wherein said line can be in the form of a tube, a pipe, a conduit, a duct, a hose, or any other form of line known in the art. Each possibility represents a separate embodiment of the present invention.

According to some embodiments, as illustrated in FIG. 1A, the enzymatic reaction assembly 100 comprises three main modules: a liquid feed 110, at least one bioreactor 130 and a product container 180. The functional and physical connectivity between the three main modules enable the method of the invention, according to some embodiments, as detailed herein. In short, according to some embodiments, a liquid feed outlet line 131, extends from the liquid feed 110 to a first junction 138 to enable fluid passing between the liquid feed 110 and the subsequent modules in the assembly 100. According to some embodiments, the liquid feed 110 acts as a container for a starting composition, which is passed through the assembly 100 and transformed into a product through the present method, specifically during step (b). According to some embodiments, the first junction 138 comprises three or more inlet and/or outlet ports. According to some embodiments, a first port of the first junction 138p1 is connected to the feed outlet line 131. According to some embodiments, a second port of the first junction 138p2 is connected to a bioreactor inlet line 131a. According to some embodiments, a third port of the first junction 138p3 is connected to a circulation line 135. According to some embodiments, the bioreactor inlet line 131a is in fluid communication with the bioreactor 130. According to some embodiments, the circulation line 135 is in fluid communication with a second junction 139. According to some embodiments, a bioreactor outlet line 134, which extends from the bioreactor 130 to the second junction 139. According to some embodiments, the second junction 139 comprises three or more inlet and/or outlet ports. According to some embodiments, a first port of the second junction 139p1 is connected to the bioreactor outlet line 134. According to some embodiments, a second port of the second junction 139p2 is connected to the circulation line 135. According to some embodiments, a third port of the second junction 139p3 is connected to product line 150. According to some embodiments, the product container 180 is in fluid communication with the product line 150.

Thus, according to some embodiments, the enzymatic reaction assembly 100 comprises liquid feed 110, at least one bioreactor 130, which contains enzymatically active composite system 140; a liquid feed outlet line 131, which extends from the liquid feed 110 to a first junction 138, wherein the first junction 138 comprises a first port connected to the feed outlet line 131, a second port connected to a bioreactor inlet line 131a and a third port connected to a circulation line 135, wherein the bioreactor inlet line 131a is in fluid communication with the bioreactor 130 and the circulation line 135 is in fluid communication with a second junction 139; a bioreactor outlet line 134, which extends from the bioreactor 130 to the second junction 139, wherein the second junction 139 comprises a first port connected to the bioreactor outlet line 134, a second port connected to the circulation line 135 and a third port connected to product line 150; and product container 180, which is in fluid communication with the product line 150.

According to some embodiments, the first junction 138 has a first open state in which the first port 138p1 and second port 138p2 are open. According to some embodiments, in the first open state of the first junction 138, liquid feed outlet line 131 is in fluid communication with the bioreactor inlet line 131a. According to some embodiments, in the first open state of the first junction 138, the third port 138p3 is closed. Specifically, the first open state of the first junction 138 can be used during step (b1) of the present method, which includes flowing the starting liquid composition from the liquid feed 110 through the liquid feed outlet line 131, the first junction 138 and the bioreactor inlet line 131a into the bioreactor 130.

According to some embodiments, the first junction 138 has a second open state in which the second port 138p2 and third port 138p3 are open. According to some embodiments, in the second open state of the first junction 138, the circulation line 135 is in fluid communication with the bioreactor inlet line 131a. According to some embodiments, in the second open state of the first junction 138, the first port 138p1 is closed. According to some embodiments, in the second open state of the first junction 138, the first port 138p1 is open. Specifically, the second open state of the first junction 138 can be used during step (b3) of the present method, which includes recirculating the liquid product composition from the bioreactor 130 through the bioreactor outlet line 134, the second junction 139, the circulation line 135, the first junction 138 and the bioreactor inlet line 131a, back into the bioreactor 130.

According to some embodiments, the first junction 138 has a third open state in which the first port 138p1 and third port 139p3 are open. According to some embodiments, in the third open state of the first junction 138, the circulation line 135 is in fluid communication with the liquid feed outlet line 131. According to some embodiments, in the third open state of the first junction 138, the second port 138p2 is closed. Specifically, the third open state of the first junction 138 can be used during step (c1) of the present method, which includes flowing an acidic solution, a basic solution or both consecutively, through the liquid feed outlet line 131, the first junction 138, the circulation line 138, the second junction 139 and the product line 150.

According to some embodiments, the second junction 139 has a first open state in which the first port 139p1 and second port 139p3 are open. According to some embodiments, in the first open state of the second junction 139, bioreactor outlet line 134 is in fluid communication with the product line 150. According to some embodiments, in the first open state of the second junction 139, the second port 139p2 is closed. Specifically, the first open state of the second junction 139 can be used during step (b4) of the present method, which includes flowing the liquid product composition from the bioreactor 130 through the bioreactor outlet line 134, the second junction 139 and the product line 150 into the product container 180.

According to some embodiments, the second junction 139 has a second open state in which the first port 139p1 and second port 139p2 are open. According to some embodiments, in the second open state of the second junction 139, the circulation line 135 is in fluid communication with the bioreactor outlet line 134. According to some embodiments, in the second open state of the second junction 139, the third port 139p3 is closed. According to some embodiments, in the second open state of the second junction 139, the third port 139p3 is open. Specifically, the second open state of the first junction 138 can be used during step (b3) of the present method, which includes recirculating the liquid product composition from the bioreactor 130 through the bioreactor outlet line 134, the second junction 139, the circulation line 135, the first junction 138 and the bioreactor inlet line 131a, back into the bioreactor 130.

According to some embodiments, the second junction 139 has a third open state in which the second port 139p2 and third port 139p3 are open. According to some embodiments, in the third open state of the second junction 139, the circulation line 135 is in fluid communication with the bioreactor outlet line 134. According to some embodiments, in the third open state of the second junction 139, the first port 139p1 is closed. Specifically, the third open state of the first junction 138 can be used during step (c1) of the present method, which includes flowing an acidic solution, a basic solution or both consecutively, through the liquid feed outlet line 131, the first junction 138, the circulation line 138, the second junction 139 and the product line 150.

According to some embodiments, the bioreactor contains an enzymatically active composite system. According to some embodiments, the bioreactor contains an enzymatically active composite system during step (a). According to some embodiments, the bioreactor contains an enzymatically active composite system during steps (a), (b) and (c).

According to some embodiments, the composite system comprises an enzymatically active agent immobilized to a support.

As used herein the terms “solid support” or “semi solid support” refer to a solidified hardened material. It is to be understood that the “semi solid” state includes hardened gels. According to some embodiments, the support is substantially water-insoluble gel at 25° C. The term “substantially insoluble” as used in this context means that at least 90% of the support, such as at least 95%, at least 98%, at least 99%, or at least 99.5% does not enter solution in water at 25° C. and pH 7.

According to some embodiments, the support is a solid support or a semi-solid support. According to some embodiments, the support is a semi-solid support. According to some embodiments, the semi-solid support comprises a harden gel.

According to some embodiments, the enzymatically active composite system comprises the enzymatically active agent is bonded to the support. According to some embodiments, the enzymatically active composite system comprises the enzymatically active agent is entrapped within the support. According to some embodiments, the enzymatically active composite system comprises the enzymatically active agent encapsulated within the support. According to some embodiments, the enzymatically active composite system comprises the enzymatically active agent is embedded in the support. According to some embodiments, the enzymatically active composite system comprises the enzymatically active agent bonded to the support, entrapped within the support, encapsulated within the support, embedded in the support or a combination thereof.

According to some embodiments, the enzymatically active composite system is in the form of beads.

According to some embodiments, the support is substantially water insoluble at 25° C. According to some embodiments, the support comprises an insoluble polymer. According to some embodiments, the support comprises an anionic polymer. According to some embodiments, the anionic polymer comprises a polymeric backbone bonded to a plurality of negatively charged functional groups. According to some embodiments, the negatively charged functional groups are selected from the group consisting of: carboxylate, sulfate, and sulfonate. According to some embodiments, the negatively charged functional groups comprise carboxylate groups. According to some embodiments, the negatively charged functional groups comprise sulfate groups. According to some embodiments, the negatively charged functional groups comprise sulfonate groups. According to some embodiments, the support further comprises potassium countercations. According to some embodiments, the potassium cations and the negatively charged functional groups are at a ratio of at least 1:2.

The phrase “the potassium cations and the negatively charged functional groups are at a ratio of at least 1:2”, as used herein is intended to mean that the total number of potassium cations is at least half of the total number of the negatively charged functional groups within the charged polymer. This includes 1.1:2, 1.25:2, 1.5:2, 1.7:2, 2:2, 2.5:2 etc. According to some embodiments, the potassium cations and the negatively charged functional groups are at a ratio of at least 1.5:2. According to some embodiments, the potassium cations and the negatively charged functional groups are at a ratio of at least 1.75:2. According to some embodiments, the potassium cations and the negatively charged functional groups are at a ratio of at least 1.9:2. According to some embodiments, the potassium cations and the negatively charged functional groups are at a ratio of about 2:2.

According to some embodiments, the anionic polymer comprises alginate or carrageenan. According to some embodiments, the anionic polymer comprises alginate. According to some embodiments, the support comprises anionic alginate and calcium countercations. According to some embodiments, the anionic polymer comprises carrageenan. According to some embodiments, the support comprises anionic carrageenan and potassium countercations.

According to some embodiments, the support comprises a combination of alginate and gellan gum or combination of carrageenan and xanthan gum. According to some embodiments, the support comprises a combination of alginate and gellan gum. According to some embodiments, the support comprises a combination of carrageenan and xanthan gum.

According to some embodiments, the enzymatically active agent comprises isolated enzymes, a plurality of microbial cells or both. According to some embodiments, the enzymatically active agent comprises a plurality of microbial cells. According to some embodiments, the enzymatically active agent comprises a plurality of non-proliferating bacterial cells, a plurality of non-proliferating fungal cells or both.

The terms “non-proliferating microbial cells” or “dead microbial cells” as used herein refers to microbial cells that are found to be non-viable when examined in a viability test (e.g., growth in nutrient agar and strain specific growth plates and/or colony forming unit (CFU) count). According to some embodiments, the microbial cells of the invention are dead microbial cells such as dead bacteria, dead fungi and/or dead yeast, which are non-proliferating microbial cells.

Obtaining the dead microbial cells/non-proliferating microbial cells while preserving their enzymatic activity can be achieved by methods known in the art.

According to some embodiments, the enzymatically active agent comprises a plurality of non-proliferating Zymomonas mobilis (ZM), non-proliferating Aspergillus japonicus (AJ), non-proliferating Aspergillus niger (AN) or a combination thereof. Each possibility represented a separate embodiment.

Presented below are specific embodiments directed to employing the method of the present invention for reducing the disaccharide content of fruit and/or vegetable juice(s). Some non-limiting embodiments directed to enzymatically active agents, which are capable of such transformation are detailed herein.

According to some embodiments, the enzymatically active agent catalyzes a transformation of monosaccharides, disaccharides, or both into at least one of at least one sugar alcohol; at least one a sugar acid; at least one oligosaccharide; at least one polysaccharide; or any combination thereof.

According to some embodiments, the enzymatically active agent reduces the content of monosaccharides, disaccharides, or both and optionally elevates one of at least one sugar alcohol; at least one sugar acid; at least one oligosaccharide; at least one polysaccharide; or any combination thereof.

According to some embodiments, the enzymatically active agent catalyzes a transformation of glucose, fructose, sucrose, or any combination thereof into at least one of sorbitol, gluconic acid, at least one fructooligosaccharides, and any combination thereof.

According to some embodiments, the enzymatically active agent reduces the content of glucose, fructose, sucrose, or any combination thereof, optionally elevates the content of at least one of sorbitol, gluconic acid, at least one fructooligosaccharides, and any combination thereof.

According to some embodiments, the enzymatically active agent comprises an enzyme selected from the group consisting of cellulose synthase, glucosyltransferase, oxidoreductases, fructosyltransferase, glucose oxidase, glucose isomerase and any combination thereof. According to some embodiments, the enzyme is glucose fructose oxidase reductase (GFOR), glucose oxidase (GOX) or sucrose fructosyltransferase (SFTase).

The non-proliferating microbial cells of the invention maintain their catalytic activity of transformation of monosaccharides, disaccharides, or both into at least one of at least one sugar alcohol; at least one a sugar acid; at least one oligosaccharide; at least one polysaccharide; or any combination thereof.

Reference is now made back to the assembly 100 employed in the method of the present invention.

According to some embodiments, the liquid feed 110 contains a starting liquid composition. It is to be understood that the starting liquid composition is transformed into a product through the present method by a chemical/biological reaction carried out within the bioreactor 130. It is further to be understood that in embodiments directed to sugar-reduction of fruit or vegetable juice, the starting liquid composition may be a raw or processed (e.g., filtered) fruit or vegetable juice.

According to some embodiments, the liquid feed 110 includes a container, a tank, or a juice press. According to some embodiments, the liquid feed 110 can be a feed tube or pipe enabling liquid transfer therethrough, wherein said feed tube is fluidly coupled to a feed container or connectable to a feed container.

As derailed herein, the liquid feed 110 is fluidly connected to the first junction 138 through the liquid feed outlet line 131. The first junction 138 is configured to monitor liquid flow from the liquid feed outlet line 131 to the bioreactor 130 through the bioreactor inlet line 131a, according to some embodiments. The first junction 138 is further configured to monitor liquid flow from the liquid feed outlet line 131 to the product container 180 through the circulation line 135, according to some embodiments. The first junction 138 is further configured to monitor liquid flow from the circulation line 135 to the bioreactor 130 through the bioreactor inlet line 131a, according to some embodiments.

According to some embodiments, the first junction 138 and/or the second junction 139 may include valves, or any other communication appliance known in the art, and combinations thereof.

According to some embodiments, the at least one bioreactor 130 comprises an inlet port 132, an outlet port 133, and at least one bioreactor wall 136 defining an internal space 137 therein. According to some embodiments, the enzymatically active composite system resides within the internal space 137. According to some embodiments, the enzymatically active composite system resides within the internal space 137 throughout steps (a)-(c) of the present method. According to some embodiments, bioreactor 130 comprises a first surface 132a comprising the inlet port 132 extending therethrough, and a second surface 133a positioned substantially parallel thereto, comprising the outlet port 133 extending therethrough. According to some embodiments, the at least one bioreactor wall 136 is positioned perpendicularly to first surface 132a and the second surface 133a. According to some embodiments, the at least one bioreactor wall 136 is extending from the first surface 132a towards the second surface 133a. According to further embodiments, the bioreactor 130 comprises a plurality of walls 136 extending from the first surface 132a towards the second surface 133a and defining the internal space 137 there-inside.

As used herein, the term “bioreactor” refers to any system, device, apparatus, or structure capable of supporting a biologically active environment and for conducting biological and/or biotechnological transformations. Bioreactors are usually cylindrical vessels, however the term is not limited to a shape and may be any closed curvilinear shape, such as a closed tube or a cylindroid, or rectilinear shape, such as a cuboid, including closed combinations of curvilinear and rectilinear shapes.

According to some embodiments, the bioreactor 130 is a closed bioreactor.

As used herein, the term “closed bioreactor” refers to a closed system which at least temporarily isolates biological media (e.g., enzymes, proliferating or non-proliferating microbial cells etc.) contained therein from the surrounding environment. It is to be understood that closed photobioreactors may include opening(s) and/or a cover, for gaining access to the medium contained therein, and are not limited to permanently sealed or closed structures. Elements, such as a cover or a port may provide reversible access to the interior of the photobioreactor, such that its closed feature may be limited to the operation period thereof (e.g., the enzymatic reaction period of step (b) and/or the disinfection of step (c1) and or (c2)).

According to some embodiments, the at least one bioreactor 130 has a three-dimensional (3D) structure, and is selected from the group consisting of: continuous stirred-tank reactor (CSTR), batch reactor, wave bioreactor, vibrating bed bioreactor, a column, and other known reactors or bioreactors in the art. Each possibility represents a separate embodiment. According to some embodiments, the at least one bioreactor 130 is a continuous stirred tank reactor (CSTR) or any other stirred reactor or bioreactor known in the art.

According to some embodiments, the enzymatic reaction assembly 100 comprises at least one circulation line 135. According to some embodiments, the circulation line 135 is positioned externally to the bioreactor 130. According to some embodiments, the at least one circulation line 135 is extending from the first junction 138 to the second junction 139. According to some embodiments, the at least one circulation line 135 is configured to circulate at least a portion of the liquid flowing through the bioreactor 130, from and into the bioreactor 130. According to some embodiments, the circulation line 135 is coupled to the bioreactor inlet line 131a through the first junction 138, and to the bioreactor outlet line 134 through the second junction 139, as illustrated for example at FIG. 1A. According to some embodiments, such configuration allows at least a portion of the liquid to be continuously circulated into the bioreactor 130 for at least one circulation cycle. As discussed with respect to step (b3), the liquid product composition may be recirculated from the bioreactor 130 through the bioreactor outlet line 134, the second junction 139, the circulation line 135, the first junction 138 and the bioreactor inlet line 131a, back into the bioreactor 130, according to some embodiments, wherein step (b3) is optional and may be performed one or more times. According to other embodiments, the circulation line 135 enables to circulate the entire content of the liquid flowing through the bioreactor 130. Advantageously, said circulation line 135 enables to circulate at least a portion of the liquid into the bioreactor 130, thus enabling to enhance the mixing and the contact time between the liquid and the enzymatically active composite system 140, to achieve optimal results.

According to some embodiments, the enzymatic reaction assembly 100 is configured to flow the starting liquid composition from liquid feed 110 through liquid feed outlet line 131, first junction 138 and bioreactor inlet line 132 into bioreactor 130. According to some embodiments, the enzymatic reaction assembly 100 is further configured to allow contacting the starting liquid composition with the enzymatically active composite system 140 in bioreactor 130, thereby inducing an enzymatic reaction, to produce a liquid product composition.

According to some embodiments, the bioreactor 130 comprises at least one filter (not shown). According to some embodiments, the at least one filter is configured to prevent from the enzymatically active composite system 140 (e.g., beads) to exit the bioreactor 130. According to further embodiments, inlet port 132 is coupled to a filter. According to further embodiments, outlet port 133 is coupled to a filter. According to some embodiments, each one of the inlet port 132 and the outlet port 133 is coupled to a separate filter. The filter(s) may contain a plurality of apertures or openings having a size which is smaller than a diameter (or a long dimension) of each bead, to prevent from the beads to pass therethrough, thereby retaining the enzymatically active composite system 140 within the bioreactor 130. Advantageously, bioreactor 130 of the present invention is configured to maintain the plurality of beads there-inside and to prevent the enzymatically active composite system 140 from exiting the bioreactor 130, and thus to allow the continuous and/or semi-continuous operation of the bioreactor 130 and the reaction between the enzymatically active composite system 140 and the liquid, without the need to exchange batches of enzymatically active composite system 140, e.g. beads, which can be time consuming and non-economic.

According to some embodiments, the bioreactor 130 may comprise at least one stirrer or impeller or any other suitable electric component, configured to stir or agitate or mix the liquid with the enzymatically active composite system 140 disposed therein. Said electric component may be coupled to an actuator. The term “actuator”, as used herein, refers to any powered actuator known in the art for providing rotational motion, such as an electric motor, a solenoid, and the like.

Reference is now made to FIG. 1B illustrating enzymatic reaction assembly 100, which comprise all the elements as described for FIG. 1A and additional elements as described herein after.

According to some embodiments, the assembly 100 further comprises a heat exchanger 120. According to some embodiments, the heat exchanger 120 is formed over the liquid feed outlet line 131. According to some embodiments, the heat exchanger 120 is functionally positioned between bioreactor 130 and liquid feed 110. According to some embodiments, the heat exchanger 120 is functionally positioned between first junction 138 and liquid feed 110. According to some embodiments, the heat exchanger 120 is functionally positioned between first junction 138 and bioreactor 130. Such construction of the heat exchanger 120 between bioreactor 130 and liquid feed 110 enables temperature adjustment in the heat exchanger 120 during step (b1). According to some embodiments, the heat exchanger 120 is configured to elevate the temperature of liquid entering thereto. Thus, according to some embodiments, in step (b1) the starting liquid composition flown from the liquid feed 110 to the bioreactor 130 may be heated by the heat exchanger 120.

According to some embodiments, the assembly 100 further comprises a heat exchanger 160. According to some embodiments, the heat exchanger 160 is formed over the product line 150. According to some embodiments, the heat exchanger 160 is functionally positioned between the bioreactor 130 and the product container 180. According to some embodiments, the heat exchanger 160 is functionally positioned between second junction 139 and product container 180. According to some embodiments, the heat exchanger 160 is functionally positioned between the second junction 139 and bioreactor 130. Such construction of the heat exchanger 160 between bioreactor 130 and product line 150 enables temperature adjustment in the heat exchanger 160 during step (b4). According to some embodiments, the heat exchanger 160 is configured to lower the temperature of liquid entering thereto. Thus, according to some embodiments, in step (b4) the liquid product composition flown from the bioreactor 130 to the product container 180 may be cooled by the heat exchanger 160.

It is to be understood that the heat exchanger 160 may be a stand-alone device or may be the same device as heat exchanger 120 (i.e., a unitary heat exchanger). In the latter option, the unitary heat exchanger is constructed so that a portion thereof is configured to elevate the temperature of the liquid feed outlet line 131 (and the liquid flowing therein) and another portion thereof is configured to lower the temperature of the product line 150 (and the liquid flowing therein).

According to some embodiments, the assembly 100 further comprises a pH adjuster 170. According to some embodiments, the pH adjuster 170 is formed over the product line 150. According to some embodiments, the pH adjuster 170 is functionally positioned between the bioreactor 130 and the product container 180. According to some embodiments, the pH adjuster 170 is functionally positioned between second junction 139 and product container 180. According to some embodiments, the pH adjuster 170 is configured to adjust the pH of aqueous compositions. According to some embodiments, the pH adjuster 170 is configured to reduce the pH of aqueous compositions. The pH adjuster 170 may be, for example an opening formed on the product line 150, through which a pH adjusting agent is inserted into the line 150. Alternatively, the pH adjuster 170 may be an automatic means for inserting such pH adjusting agent into the line 150. The opening may be, for example a conduit connected or connectable to a feed of the product line 150. The pH adjusting agent, according to some embodiments, may be an aqueous composition. According to some embodiments, the aqueous composition is acidic. Such construction of the pH adjuster 170 between bioreactor 130 and product container 180 enables pH adjustment during step (b4). Thus, according to some embodiments, in step (b4) the liquid product composition flown from the bioreactor 130 to the product container 180 may be acidified by the pH adjuster 170.

According to some embodiments, the assembly 100 further comprises a sampling port 103. According to some embodiments, the sampling port 103 is formed over the product line 150. According to some embodiments, the sampling port 103 is functionally positioned between the bioreactor 130 and the product container 180. According to some embodiments, the sampling port 103 is functionally positioned between second junction 139 and product container 180. The sampling port 103 may be, for example an opening formed on the product line 150, through which an aliquot of a liquid flowing in product line 150 may be sampled. Alternatively, or in addition, according to some embodiments, sampling port 103 may include a sensor configured to measure a parameter of the liquid flowing in product line 150. Such construction of the sampling port 103 between bioreactor 130 and product container 180 sampling of the liquid product composition during step (b4). Thus, according to some embodiments, in step (b4) the liquid product composition flown from the bioreactor 130 to the product container 180 may sampled by the sampling port 103.

Reference is now made to FIG. 1C depicting enzymatic reaction assembly 100, which comprise all the elements as described in FIGS. 1A-1B and additional element as described herein after.

According to some embodiments, the assembly 100 further comprises a pH adjuster 122. According to some embodiments, the pH adjuster 122 is formed over the liquid feed outlet line 131. According to some embodiments, the pH adjuster 122 is functionally positioned between the bioreactor 130 and the liquid feed 110. According to some embodiments, the pH adjuster 122 is functionally positioned between first junction 138 and bioreactor 130. According to some embodiments, the pH adjuster 122 is configured to adjust the pH of aqueous compositions. According to some embodiments, the pH adjuster 122 is configured to elevate the pH of aqueous compositions. The pH adjuster 122 may be, for example an opening formed on the liquid feed outlet line 131, through which a pH adjusting agent is inserted into the line 131. Alternatively, the pH adjuster 122 may be an automatic means for inserting such pH adjusting agent into the line 131. The opening may be, for example a conduit connected or connectable to a feed of the line 131. The pH adjusting agent, according to some embodiments, may be an aqueous composition. According to some embodiments, the aqueous composition is basic. Such construction of the pH adjuster 122 between bioreactor 130 and liquid feed 110 enables pH adjustment during step (b1). Thus, according to some embodiments, in step (b1) the starting liquid composition flown from the liquid feed 110 to the bioreactor 130 may be basified by the pH adjuster 122.

According to some embodiments, the pH adjuster 122 comprises an ion exchanger. According to some embodiments, the pH adjuster 122 is an ion exchanger. According to some embodiments, the ion exchanger comprises an ion exchange agent. The ion exchange agent, according to some embodiments, may be an ion exchange resin.

Ion exchange is an inverse chemical reaction where ions in a fluid medium (such as the liquid composition) are exchanged for similarly charged ions that are bound to immobile solid particles that are insoluble or substantially insoluble in the fluid medium. The term “ion exchange resin” as used herein refers to all such materials. Due to the crosslinkability of the polymer support to which the ion exchange groups are attached, the resin becomes insoluble. Ion exchange resins are classified as cation exchangers or anion exchangers. Cation exchangers have positively charged mobile ions available for exchange, typically metal ions such as protons, potassium, or sodium cations. Anion exchangers have negatively charged exchangeable ions, typically hydroxide ions.

According to some embodiments, the pH adjuster 122 comprises an ion exchange column, which contains ion exchange resin therein. According to some embodiments, the ion exchange resin is a cation exchange resin. According to some embodiments, the ion exchange resin is an anion exchange resin. According to some embodiments, upon passing the juice through the ion exchange column, the pH of the juice is reduced to bring the juice to its starting pH.

According to some embodiments, at least one of the circulation line 135, liquid feed outlet line 131, bioreactor outlet line 134, combinations thereof, or any other portion or line of assembly 100, is coupled or is in fluid communication with at least one pump. According to some embodiments, the circulation line 135 is coupled or is in fluid communication with at least one pump 124. According to some embodiments, the bioreactor inlet line 131a and/or bioreactor outlet line 134 is coupled or is in fluid communication with at least one pump 124. The pump(s) are configured to pump or convey the liquid through the enzymatic reaction assembly, at specific flow rate(s). Sown in FIG. 1C are two optional, non-limiting positions of the pump 124. Pump 124a, according to some embodiments, is functionally positioned over circulation line 135. Such configuration enables pumping liquid during step (b2), (b3) (b4) and/or (c1). Each possibility represents a separate embodiment of the invention. Pump 124b, according to some embodiments, is functionally positioned over liquid feed outlet line 131. Such configuration enables pumping liquid during step (b1), (b2), (b3), (b4) and/or (c1). Each possibility represents a separate embodiment of the invention.

According to some embodiments, assembly 100 comprises at least one control system 190 (see FIG. 1C) which is in operative communication with various assembly components such as pump(s), temperature adjusting apparatus(es), and the like, in order to adjust their operation based on various system requirements and user demand. In further embodiments, the control system 190 can automatically receive sensor data or readings from any sensor(s) coupled to the assembly 100, and adjust the operation of various assembly components based on said sensor data. For example, if the °Bx content of the liquid product leaving the bioreactor is higher than a certain threshold, the control system can adjust the operation of the pump 124.

According to some embodiments, the control system 190 comprises a computer system including, for example, a processor coupled to a tangible, non-transitory memory. According to some embodiments, the control system 190 is in the form of an operating hand-controller (e.g., cell phone, smartphone, laptop, tablet, etc.) and/or a control panel comprising one or more screens (e.g., touchscreens). The screens or touchscreens can include multiple color screens which may provide visualization of data coming from the sensors or from other system components. According to some embodiments, the control system 190 further comprises connection to Ethernet for remote monitoring of process parameters. According to some embodiments, the control system 190 comprises one or more elements selected from a processor (e.g., a central processing unit (CPU)), a graphics processing unit (GPU, etc.), computer-readable storage device (e.g., main memory, static memory, etc.), or combinations thereof, wherein said elements can communicate with each other via a wired or wireless communication.

Specific reference is now made to step (b) of the present method. According to some embodiments, the present method includes step (b) of performing an enzymatic reaction. According to some embodiments, as detailed herein step (b) is divided into steps (b1), (b2), (b3) and (b4), wherein step (b3) is optional. According to some embodiments, steps (b1), (b2) and (b4) or steps (b1), (b2), (b3) and (b4) are performed according to their numeric order. According to some embodiments, step (b) is performed a plurality of times.

According to some embodiments, step (b1) to (b4) are performed simultaneously. According to some embodiments, step (b1), (b2) and (b4) are performed simultaneously.

According to some embodiments, each one of steps (b1), (b2), (b3) and (b4) comprises flowing a liquid through the bioreactor assembly 100. According to some embodiments, flowing the liquid through the bioreactor assembly 100 is performed at a flow rate of 0.5 L/hr to 3 L/hr, including each value and sub-range within the specified range. According to some embodiments flowing a liquid through the bioreactor assembly 100 is at a flow rate selected from the range of 0.1-4 L/hr, alternatively 0.8-4 L/hr, or optionally 0.1-2 L/hr. Each possibility represents a different embodiment. According to some embodiments flowing a liquid through the bioreactor assembly 100 is at a flow rate of about 1.2 L/hr.

Specific reference is now made to step (b1) of the present method. According to some embodiments, the present method includes step (b1) of flowing the starting liquid composition from the liquid feed 110 through the liquid feed outlet line 131, the first junction 138 and the bioreactor inlet line 131a into the bioreactor 130. According to some embodiments the flowing of step (b1) is at a flow rate of about 1.2 L/hr.

According to some embodiments, during step (b1) the first port 138p1 of the first junction 138 is open. According to some embodiments, during step (b1) the second port 138p2 of the first junction 138 is open.

According to some embodiments, during step (b1) the first junction 138 is at its first open state. According to some embodiments, step (b1) comprises switching the first junction 138 to the first open state. According to some embodiments, during step (b1) the third port 138p3 of the first junction 138 is closed. Thus, according to some embodiments, step (b1) comprises preventing fluid flow from the liquid feed outlet line 131 to the circulation line 135. According to some embodiments, step (b1) comprises preventing fluid flow from the liquid feed 110 to the circulation line 135. Specifically, allowing liquid flow from the liquid feed 110 through the circulation line 135 may result in portions of the starting liquid composition to flow directly (i.e., unreacted) to the product container 180, according to some embodiments. Therefore, according to some embodiments, it is beneficial to prevent such flow during initial cycle of step (b).

As detailed herein, steps (b1)-(b4) may be performed simultaneously, according to some embodiments. In such case, the flow of starting liquid composition may be enabled through both the bioreactor inlet line 131a and the circulation line 135. Thus, according to some embodiments, during step (b1) the first junction 138 is at its second open state. According to some embodiments, step (b1) comprises switching the first junction 138 to the second open state. According to some embodiments, during step (b1) the third port 138p3 of the first junction 138 is open. Thus, according to some embodiments, step (b1) comprises allowing fluid flow from the liquid feed outlet line 131 to the circulation line 135. According to some embodiments, step (b1) comprises allowing fluid flow from the liquid feed 110 to the circulation line 135.

According to some embodiments, during step (b1) the first port 139p1 of the second junction 139 is open. According to some embodiments, during step (b1) the second port 139p2 of the second junction 139 is open or closed. Each possibility represents a separate embodiment of the invention. According to some embodiments, during step (b1) the third port 139p3 of the second junction 139 is open.

As detailed herein, steps (b1)-(b4) may be performed simultaneously, according to some embodiments. In such case, the flow of starting liquid composition may be enabled through both the bioreactor outlet line 134, circulation line 135 and product line 150. Thus, according to some embodiments, during step (b1) the second junction 139 is at its second open state, wherein the third port 139p3 is open. According to some embodiments, step (b1) comprises switching the second junction 139 to the second open state. According to some embodiments, during step (b1) the third port 139p3 of the second junction 139 is open.

As described herein, according to some embodiments, the assembly 100 comprises a heat exchanger 120, formed over the liquid feed outlet line 131. According to some embodiments, step (b1) further comprises adjusting the temperature of the starting liquid composition. According to some embodiments, step (b1) comprises adjusting the temperature of the starting liquid composition using the heat exchanger 120. According to some embodiments, adjusting the temperature of the starting liquid composition in step (b1) entails elevating the temperature of the starting liquid composition. According to some embodiments, step (b1) comprises elevating the temperature of the starting liquid composition by at least 5° C., at least 10° C. or at least 15° C. Each possibility represents a separate embodiment of the invention. According to some embodiments, step (b1) comprises elevating the temperature of the starting liquid composition by no more than 100° C., no more than 50° C. or no more than 35° C. Each possibility represents a separate embodiment of the invention. According to some embodiments, step (b1) comprises elevating the temperature of the starting liquid composition by 5° C. to 30° C. or 10° C. to 25° C. Each possibility represents a separate embodiment of the invention. According to some embodiments, step (b1) comprises elevating the temperature of the starting liquid composition to about 25° C. According to some embodiments, step (b1) comprises elevating the temperature of the starting liquid composition to at least 15° C. According to some embodiments, step (b1) comprises elevating the temperature of the starting liquid composition to a range of 15° C. to 75° C., including each value and sub-range within the specified range.

As described above, according to some embodiments, the assembly 100 comprises a pH adjuster 122. According to some embodiments, step (b1) further comprises adjusting the pH of the starting liquid composition. According to some embodiments, step (b1) comprises adjusting the pH of the starting liquid composition using the pH adjuster 122. According to some embodiments, adjusting the pH of the starting liquid composition in step (b1) entails elevating the pH of the starting liquid composition. According to some embodiments, step (b1) comprises elevating the pH of the starting liquid composition by at least 1, at least 2, or at least 3 pH units. Each possibility represents a separate embodiment of the invention. According to some embodiments, step (b1) comprises elevating the pH of the starting liquid composition by no more than 8, no more than 7 or no more than 6 pH units. Each possibility represents a separate embodiment of the invention. According to some embodiments, step (b1) comprises elevating the pH of the starting liquid composition by 3 to 5 pH units. According to some embodiments, step (b1) comprises elevating the pH of the starting liquid composition to about 7. According to some embodiments, step (b1) comprises elevating the pH of the starting liquid composition to at least 5.5. According to some embodiments, step (b1) comprises elevating the pH of the starting liquid composition to a range of 5.5 to 8, including each value and sub-range within the specified range. According to some embodiments, adjusting the pH of the starting liquid composition in step (b1) comprises contacting the starting liquid composition with a pH adjusting agent. According to some embodiments, the pH adjusting agent comprises a base. According to some embodiments, the pH adjusting agent comprises an alkali base. According to some embodiments, the alkali base is selected from the group consisting of: potassium hydroxide, sodium hydroxide, potassium carbonate, sodium carbonate, potassium bicarbonate, sodium bicarbonate, potassium phosphate, sodium phosphate and a combination thereof. Each possibility represents a separate embodiment of the invention. According to some embodiments, the alkali base comprises a potassium salt. According to some embodiments, the alkali base is selected from the group consisting of: potassium hydroxide, potassium carbonate, potassium bicarbonate. According to some embodiments, the alkali base comprises potassium hydroxide. According to some embodiments, the pH adjusting agent comprises an aqueous solution of the pH adjusting agent.

According to some embodiments, flowing the starting liquid composition from the liquid feed 110 through the liquid feed outlet line 131, the first junction 138 and the bioreactor inlet line 131a into the bioreactor 130 in step (b1) is performed using the at least one pump 124.

Specific reference is now made to step (b2) of the present method. According to some embodiments, the present method includes step (b2) of contacting the starting liquid composition with the enzymatically active composite system in the bioreactor 130, thereby inducing an enzymatic reaction, to produce a liquid product composition.

The method of the present invention advantageously enables to produce a liquid product composition in a continuous or semi-continuous manner, according to some embodiments. This, according to some embodiments, is enabled by the particular sequence of disinfection steps (c1) and (c2) which does not require frequent replacement of the enzymatically active composite system within the bioreactor 130.

As used herein, the term “continuous or semi-continuous” production protocol refers to a range of production methods, in which the product is formed without substantial pauses for maintenance of the production means. In the context of bioreactions, which involve enzymatically active composite systems, continuous or semi-continuous methods avoid frequent replacement of said composites. Typically, such protocols are referred as clean-in-place (CIP). Thus, according to some embodiments, the present invention provides a method for performing an enzymatic reaction and CIP of the assembly and enzymatically active composite system used for carrying out said reaction.

According to some embodiments, the present method involves continuous or semi-continuous production of the liquid product composition through a continuous or semi-continuous flow of the starting liquid composition into the bioreactor 130 and its continuous or semi-continuous contacting with the enzymatically active composite system 140. Thus, according to some embodiments, step (b2) involves flowing the starting liquid composition through the bioreactor 130 and through its contacting with the enzymatically active composite system 140.

According to some embodiments the flowing of step (b2) is at a flow rate of about 1.2 L/hr. According to some embodiments, the flowing is performed using the at least one pump 124.

According to some embodiments, step (b2) is performed at a temperature of about 25° C. According to some embodiments, step (b2) is performed at a temperature of at least 15° C. According to some embodiments, step (b2) is performed at a temperature in a range of 15° C. to 75° C., including each value and sub-range within the specified range.

According to some embodiments, step (b2) is performed at a pH of about 7. According to some embodiments, step (b2) is performed at a pH of least 5.5. According to some embodiments, step (b2) is performed at a pH in a range of 5.5 to 8, including each value and sub-range within the specified range.

As detailed herein, the present method may be used for reducing the sugar content of fruit and/or vegetable juices. According to some embodiments, the enzymatic reaction of step (b2) entails reducing the content of at least one mono- and/or disaccharide. Thus, according to some embodiments, the liquid product composition has lower content of at least one mono- and/or disaccharides compared to the starting liquid composition. According to some embodiments, the mono- and/or disaccharides comprise glucose, sucrose and/or fructose. According to some embodiments, the enzymatic reaction of step (b2) entails forming dietary fiber. Thus, according to some embodiments, the liquid product composition has higher content of dietary fiber compared to the starting liquid composition. According to some embodiments, the dietary comprises a fructooligosaccharide (FOS).

According to some embodiments, during step (b2) the second port 138p2 of the first junction 138 is open.

According to some embodiments, during step (b2) the first port 138p1 of the first junction 138 is open. According to some embodiments, during step (b2) the first port 138p1 of the first junction 138 is closed. According to some embodiments, during step (b2) the first port 138p1 of the first junction 138 is open and closed alternately.

According to some embodiments, during step (b2) the third port 138p3 of the first junction 138 is open. According to some embodiments, during step (b2) the third port 138p3 of the first junction 138 is closed. According to some embodiments, during step (b2) the third port 138p3 of the first junction 138 is open and closed alternately.

As detailed herein, steps (b1)-(b4) may be performed simultaneously, according to some embodiments. In such case, the flow of starting liquid composition may be enabled through both the bioreactor inlet line 131a and the circulation line 135 and the reaction of step (b2) is performed simultaneously. Thus, according to some embodiments, during step (b2) the first junction 138 is at its second open state. According to some embodiments, step (b2) comprises switching the first junction 138 to the second open state. According to some embodiments, during step (b2) the third port 138p3 of is open. Thus, according to some embodiments, step (b2) comprises allowing fluid flow into the bioreactor 130.

According to some embodiments, during step (b2) the first port 139p1 of the second junction 139 is open.

According to some embodiments, during step (b2) the second port 139p2 of the second junction 139 is open. According to some embodiments, during step (b2) the second port 139p2 of the second junction 139 is closed. According to some embodiments, during step (b2) the second port 139p2 of the second junction 139 is open and closed alternately.

According to some embodiments, during step (b2) the third port 139p3 of the second junction 139 is open. According to some embodiments, during step (b2) the third port 139p3 of the second junction 139 is closed. According to some embodiments, during step (b2) the third port 139p3 of the second junction 139 is open and closed alternately.

Steps (b2) and (b4) may be performed simultaneously, according to some embodiments. Also, steps (b1), (b2) and (b4) may be performed simultaneously, according to some embodiments. In such cases, flow of starting liquid composition may be enabled through both the bioreactor outlet line 134 and product line 150, while the reaction within the bioreactor 130 is carried out. Thus, according to some embodiments, during step (b2) the second junction 139 is at its first open state. According to some embodiments, step (b2) comprises switching the second junction 139 to the first open state. According to some embodiments, during step (b2) the second port 139p2 of the second junction 139 is closed.

As steps (b1)-(b4) may be performed simultaneously, according to some embodiments, the flow of starting liquid composition may be enabled through both the bioreactor outlet line 134, circulation line 135, while the reaction within the bioreactor 130 is carried out. Thus, according to some embodiments, during step (b2) the second junction 139 is at its second open state. According to some embodiments, during step (b2) the second junction 139 is at its second open state, wherein the third port 139p3 is open. In such case, the liquid product composition is flowing to the product container 180 simultaneously with the reaction of step (b2) and the circulation of step (b3). According to some embodiments, during step (b2) the second junction 139 is at its second open state, wherein the third port 139p3 is closed. In such case, the circulation of step (b3) is carried out simultaneously with the reaction of step (b2).

Specific reference is now made to step (b3) of the present method. According to some embodiments, the present method includes optional step (b3) of recirculating the liquid product composition from the bioreactor 130 through the bioreactor outlet line 134, the second junction 139, the circulation line 135, the first junction 138 and the bioreactor inlet line 131a, back into the bioreactor 130. As detailed herein step (b3) is optional. According to some embodiments, step (b3) is not performed. According to some embodiments, step (b3) is performed. According to some embodiments, step (b3) is performed one or more times. According to some embodiments, step (b3) is performed a plurality of times. According to some embodiments, step (b3) is repeated a plurality of times between step (b2) and step (b4). According to some embodiments, step (b3) is performed continuously. According to some embodiments, step (b3) is performed continuously and simultaneously with step (b1). According to some embodiments, step (b3) is performed continuously and simultaneously with step (b2). According to some embodiments, step (b3) is performed continuously and simultaneously with step (b4).

The term “plurality” as used herein means at least two.

Advantageously, the circulating of step (b3) during the enzymatic reaction period of step (b2) beneficially enables to enhance the mixing and monitor the contact duration between a liquid and the enzymatically active composite system 140, to achieve optimal results.

According to some embodiments the recirculating of step (b3) is at a flow rate of about 1.2 L/hr.

According to some embodiments, recirculating the liquid product composition from the bioreactor 130 through the bioreactor outlet line 134, the second junction 139, the circulation line 135, the first junction 138 and the bioreactor inlet line 131a, back into the bioreactor 130 in step (b3) is performed using the at least one pump 124.

As detailed herein, according to some embodiments, the bioreactor 130 may comprise at least one filter, configured to prevent from the enzymatically active composite system 140 (e.g., beads) to exit the bioreactor 130. According to some embodiments, step (b3) comprises filtering the enzymatically active composite system 140. According to some embodiments, step (b3) comprises flowing the liquid product composition from the bioreactor 130 to the bioreactor outlet line 134, while filtering the enzymatically active composite system 140, so that it is maintained within the bioreactor 130.

According to some embodiments, during step (b3) the second port 138p2 of the first junction 138 is open.

According to some embodiments, during step (b3) the third port 138p3 of the first junction 138 is open.

According to some embodiments, during step (b3) the first port 138p1 of the first junction 138 is open. According to some embodiments, during step (b3) the first port 138p1 of the first junction 138 is closed. According to some embodiments, during step (b3) the first port 138p1 of the first junction 138 is open and closed alternately.

As detailed herein, steps (b2)-(b3) may be performed simultaneously, according to some embodiments. In such case, the flow of liquid product composition may be enabled from the circulation line 135 through the bioreactor inlet line 131a to the bioreactor 130, and the reaction of step (b2) is performed simultaneously. Thus, according to some embodiments, during step (b3) the first junction 138 is at its third open state. According to some embodiments, step (b3) comprises switching the first junction 138 to the third open state.

As detailed herein, steps (b1)-(b4) may be performed simultaneously, according to some embodiments. In such case, (i) the flow of starting liquid composition may be enabled through the bioreactor inlet line 131a into the bioreactor 130; (ii) the reaction of step (b2) is performed; (iii) the flow of liquid product composition may be enabled from the bioreactor outlet line 134 into the circulation line 135 and back into the bioreactor 130 through the bioreactor inlet line 131a; (iv) the flow of liquid product composition may be enabled from the bioreactor outlet line 134 into the product line 150; simultaneously. Thus, according to some embodiments, during step (b3) the first junction 138 is at its second open state, wherein the second port 138p2 is open. According to some embodiments, step (b2) comprises switching the first junction 138 to the second open state, wherein the second port 138p2 is open.

According to some embodiments, during step (b3) the first port 139p1 of the second junction 139 is open.

According to some embodiments, during step (b3) the second port 139p2 of the second junction 139 is open.

According to some embodiments, during step (b3) the third port 139p3 of the second junction 139 is open. According to some embodiments, during step (b3) the third port 139p3 of the second junction 139 is closed. According to some embodiments, during step (b3) the third port 139p3 of the second junction 139 is open and closed alternately.

According to some embodiments, step (b3) comprises switching the second junction 139 to the second open state. According to some embodiments, step (b3) comprises switching the second junction 139 to the second open state.

With respect to the second junction 139, steps (b2)-(b3) may be performed simultaneously, according to some embodiments. In such case, the flow of liquid product composition may be enabled from the bioreactor 130 through the bioreactor outlet line 134 to the circulation line 135, and the reaction of step (b2) is performed simultaneously. Thus, according to some embodiments, during step (b3) the second junction 139 is at its second open state. According to some embodiments, step (b3) comprises switching the second junction 139 to the second open state, wherein the third port 139p3 is closed.

Also, steps (b1)-(b4) may be performed simultaneously, according to some embodiments. In such case, (i) the flow of starting liquid composition may be enabled through the bioreactor inlet line 131a into the bioreactor 130; (ii) the reaction of step (b2) is performed; (iii) the flow of liquid product composition may be enabled from the bioreactor outlet line 134 into the circulation line 135 and back into the bioreactor 130 through the bioreactor inlet line 131a; (iv) the flow of liquid product composition may be enabled from the bioreactor outlet line 134 into the product line 150; simultaneously. Thus, according to some embodiments, during step (b3) the second junction 139 is at its second open state, wherein the third port 139p3 is open. According to some embodiments, step (b3) comprises switching the second junction 139 to the second open state, wherein the third port 139p3 is open.

Specific reference is now made to step (b4) of the present method. According to some embodiments, the present method includes optional step (b4) of flowing the liquid product composition from the bioreactor 130 through the bioreactor outlet line 134, the second junction 139 and the product line 150 into the product container 180.

According to some embodiments the flowing of step (b4) is at a flow rate of about 1.2 L/hr. According to some embodiments, flowing the liquid product composition from the bioreactor 130 through the bioreactor outlet line 134, the second junction 139 and the product line 150 into the product container 180 is performed using the at least one pump 124.

As detailed herein, according to some embodiments, the bioreactor 130 may comprise at least one filter, configured to prevent from the enzymatically active composite system 140 (e.g., beads) to exit the bioreactor 130. According to some embodiments, step (b4) comprises filtering the enzymatically active composite system 140. According to some embodiments, step (b4) comprises flowing the liquid product composition from the bioreactor 130 through the bioreactor outlet line 134, the second junction 139 and the product line 150 into the product container 180, while filtering the enzymatically active composite system 140, so that it is maintained within the bioreactor 130.

As described herein, according to some embodiments, the assembly 100 comprises a heat exchanger 160, formed over the product line 150. According to some embodiments, step (b4) further comprises adjusting the temperature of the liquid product composition. According to some embodiments, step (b4) comprises adjusting the temperature of the liquid product composition using the heat exchanger 160. According to some embodiments, adjusting the temperature of the liquid product composition in step (b4) entails reducing the temperature of the liquid product composition. According to some embodiments, step (b4) comprises reducing the temperature of the liquid product composition by at least 5° C., at least 10° C. or at least 15° C. Each possibility represents a separate embodiment of the invention. According to some embodiments, step (b4) comprises reducing the temperature of the liquid product composition by no more than 100° C., no more than 50° C. or no more than 35° C. Each possibility represents a separate embodiment of the invention. According to some embodiments, step (b4) comprises reducing the temperature of the liquid product composition by 5° C. to 30° C. or 10° C. to 25° C. Each possibility represents a separate embodiment of the invention. According to some embodiments, step (b4) comprises reducing the temperature of the liquid product composition to 2° C. to 15° C., including each value and sub-range within the specified range.

As described above, according to some embodiments, the assembly 100 comprises a pH adjuster 170. According to some embodiments, step (b4) further comprises adjusting the pH of the liquid product composition. According to some embodiments, step (b4) comprises adjusting the pH of the liquid product composition using the pH adjuster 170. According to some embodiments, adjusting the pH of the liquid product composition in step (b4) entails reducing the pH of the liquid product composition. According to some embodiments, step (b4) comprises reducing the pH of the liquid product composition by at least 1, at least 2, or at least 3 pH units. Each possibility represents a separate embodiment of the invention. According to some embodiments, step (b4) comprises reducing the pH of the liquid product composition by no more than 8, no more than 7 or no more than 6 pH units. Each possibility represents a separate embodiment of the invention. According to some embodiments, step (b4) comprises reducing the pH of the liquid product composition by 3 to 5 pH units. According to some embodiments, step (b4) comprises reducing the pH of the liquid product composition to the range of 2 to 5, including each value and sub-range within the specified range. According to some embodiments, step (b4) comprises reducing the pH of the liquid product composition to at no more than 5. According to some embodiments, adjusting the pH of the liquid product composition in step (b4) comprises contacting the liquid product composition with a pH adjusting agent. According to some embodiments, the pH adjusting agent comprises an acid. According to some embodiments, adjusting the pH of the liquid product composition in step (b4) comprises contacting the liquid product composition with an aqueous solution of the pH adjusting agent.

According to some embodiments, during step (b4) the first port 138p1 of the first junction 138 is open.

According to some embodiments, during step (b4) the second port 138p2 of the first junction 138 is open.

According to some embodiments, during step (b4) the third port 138p3 of the first junction 138 is open. According to some embodiments, during step (b4) the third port 138p3 of the first junction 138 is closed. According to some embodiments, during step (b4) the third port 138p3 of the first junction 138 is open and closed alternately.

According to some embodiments, steps (b1), (b2) and (b4) are performed simultaneously. According to some embodiments, during step (b4) the first junction 138 is at its first open state. According to some embodiments, step (b4) comprises switching the first junction 138 to the first open state. According to some embodiments, during step (b4) the third port 138p3 of the first junction 138 is closed. Thus, according to some embodiments, step (b4) comprises preventing fluid flow from the liquid feed outlet line 131 to the circulation line 135. According to some embodiments, step (b4) comprises preventing fluid flow from the liquid feed 110 to the circulation line 135. Specifically, allowing liquid flow from the liquid feed 110 through the circulation line 135 may result in portions of the starting liquid composition to flow directly (i.e., unreacted) to the product container 180, according to some embodiments. Therefore, according to some embodiments, it is beneficial to prevent such flow during initial cycle of step (b).

As detailed herein, steps (b1)-(b4) may be performed simultaneously, according to some embodiments. In such case, the flow of liquid product composition may be enabled from the circulation line 135 to the bioreactor inlet line 131a. Thus, according to some embodiments, during step (b4) the first junction 138 is at its second open state, wherein the second port 138p2 is open. According to some embodiments, step (b4) comprises switching the first junction 138 to the second open state, wherein the second port 138p2 is open. According to some embodiments, during step (b4) the third port 138p3 of the first junction 138 is open.

According to some embodiments, during step (b4) the first port 139p1 of the second junction 139 is open.

According to some embodiments, during step (b4) the second port 139p2 of the second junction 139 is open. According to some embodiments, during step (b4) the second port 139p2 of the second junction 139 is closed. According to some embodiments, during step (b4) the second port 139p2 of the second junction 139 is open and closed alternately.

According to some embodiments, during step (b4) the third port 139p3 of the second junction 139 is open.

Steps (b2) and (b4) may be performed simultaneously, according to some embodiments. Also, steps (b1), (b2) and (b4) may be performed simultaneously, according to some embodiments. In such cases, flow of liquid product composition may be enabled through both the bioreactor outlet line 134 and product line 150, while the reaction within the bioreactor 130 is carried out. Thus, according to some embodiments, during step (b4) the second junction 139 is at its first open state. According to some embodiments, step (b4) comprises switching the second junction 139 to the first open state. According to some embodiments, during step (b4) the second port 139p2 of the second junction 139 is closed.

Also, steps (b1)-(b4) may be performed simultaneously, according to some embodiments. In such case, (i) the flow of starting liquid composition may be enabled through the bioreactor inlet line 131a into the bioreactor 130; (ii) the reaction of step (b2) is performed; (iii) the flow of liquid product composition may be enabled from the bioreactor outlet line 134 into the circulation line 135 and back into the bioreactor 130 through the bioreactor inlet line 131a; (iv) the flow of liquid product composition may be enabled from the bioreactor outlet line 134 into the product line 150; simultaneously. Thus, according to some embodiments, during step (b4) the second junction 139 is at its second open state, wherein the third port 139p3 is open. According to some embodiments, step (b4) comprises switching the second junction 139 to the second open state, wherein the third port 139p3 is open.

Specific reference is now made to step (c) of the present method. According to some embodiments, the present method includes step (c) of performing a disinfection. As detailed herein step (c) is divided into steps (c1) and (c2), according to some embodiments, wherein steps (c1) and (c2) are performed at any order. According to some embodiments, step (c1) precedes step (c2). According to some embodiments, step (c2) precedes step (c1).

According to some embodiments, the enzymatically active composite system 140 is contained within the bioreactor 130 throughout the disinfection.

According to some embodiments, the method of the present invention includes separation of the disinfection protocol into two separate steps; step (c1) directed to the disinfection of the different elements of the assembly 100; and step (c2) directed to the disinfection of the bioreactor 130 and enzymatically active composite system 140 contained therein. Advantageously, such separation allows to employ suitable disinfectants to each of the steps. Specifically, manufacturing assemblies, such as assembly 100 are typically disinfected using acids and/or bases which are not suitable for contacting biologically active matter, such as the enzymatically active composite system 140.

According to some embodiments, steps (c1) and (c2) may be performed simultaneously or sequentially. Each possibility represents a separate embodiment of the invention.

According to some embodiments, steps (b) and (c) are performed sequentially.

The terms “disinfection” and “disinfecting” should be interpreted broadly as a method, process or step that reduces the total amount of biological contaminants on, in or within a device or matter. Such contaminant may include, e.g., bacteria, viruses, fungi, parasites, and biological secretion derived from such organisms.

Specific reference is now made to step (c1) of the present method. According to some embodiments, the present method includes step (c1) of flowing an acidic solution, flowing a basic solution or flowing both consecutively, through the liquid feed outlet line 131, the first junction 138, the circulation line 135, the second junction 139 and the product line 150, thereby washing a portion of the assembly 100, which does not include the bioreactor 130.

According to some embodiments, step (c1) includes flowing the acidic solution through the liquid feed outlet line 131, the first junction 138, the circulation line 135, the second junction 139 and the product line 150. According to some embodiments, step (c1) includes flowing the basic solution through the liquid feed outlet line 131, the first junction 138, the circulation line 135, the second junction 139 and the product line 150. According to some embodiments, step (c1) includes flowing the basic and acidic solutions consecutively through the liquid feed outlet line 131, the first junction 138, the circulation line 135, the second junction 139 and the product line 150.

The terms “consecutively” and “sequentially” should be interpreted broadly to refer to the two flowing steps one immediately after the other, one after the other with a break or any one after the other, with another disinfection or any other step therebetween.

According to some embodiments, step (c1) comprises washing the portion of the assembly with an aqueous basic solution. According to some embodiments, the basic solution has pH in the range of 10 to 14, including each value and sub-range within the specified range. According to some embodiments, the basic solution has pH in the range of 10 to 12, 11 to 13 or 12 to 14. Each possibility represents a separate embodiment of the invention. According to some embodiments, the basic solution has pH of at least 10. According to some embodiments, the basic solution has pH of at least 11. According to some embodiments, the basic solution has pH of about 12. According to some embodiments, the basic solution comprises NaOH, KOH or both. According to some embodiments, the basic solution is an aqueous solution. According to some embodiments, the NaOH, KOH or both is at a concentration of about 1%, 1.5%, 2%, 2.5%, 3% w/w. According to some embodiments, washing the portion of the assembly with an aqueous basic solution is performed at a temperature in the range of 25° C. to 100° C., including each value and sub-range within the specified range. According to some embodiments, washing the portion of the assembly with an aqueous basic solution is performed at a temperature in the range of 50° C. to 100° C.

According to some embodiments, step (c1) comprises washing the portion of the assembly with an aqueous acidic solution. According to some embodiments, the acidic solution has pH in the range of 0 to 3, including each value and sub-range within the specified range. According to some embodiments, the acidic solution has pH in the range of 1 to 2, 2 to 3 or 2 to 4. Each possibility represents a separate embodiment of the invention. According to some embodiments, the acidic solution comprises hydrochloric acid, sulfuric acid, phosphoric acid or nitric acid. Each possibility represents a separate embodiment of the invention. According to some embodiments, the acidic solution is an aqueous solution. According to some embodiments, washing the portion of the assembly with an aqueous acidic solution is performed at a temperature in the range of 25° C. to 100° C., including each value and sub-range within the specified range. According to some embodiments, washing the portion of the assembly with an aqueous acidic solution is performed at a temperature in the range of 50° C. to 100° C.

According to some embodiments, step (c1) comprises (ia) washing the portion of the assembly with the aqueous basic solution, (iia) washing the portion of the assembly with a pH neutral aqueous solution, and (iiia) washing the portion of the assembly with the aqueous acidic solution; or (ib) washing the portion of the assembly with the aqueous acidic solution, (iib) washing the portion of the assembly with a pH neutral aqueous solution, and (iiib) washing the portion of the assembly with the aqueous basic solution.

As referred to in the present disclosure neutral pH means a pH in the range of 5 to 10. According to some embodiments, washing the portion of the assembly with the pH neutral aqueous solution is performed at a temperature in the range of 25° C. to 100° C., including each value and sub-range within the specified range.

According to some embodiments, during step (c1) the first port 138p1 of the first junction 138 is open.

According to some embodiments, during step (c1) the second port 138p2 of the first junction 138 is closed.

According to some embodiments, during step (c1) the third port 138p3 of the first junction 138 is open.

Thus, according to some embodiments, during step (c1) the first junction is in its second open state, wherein the second port 138p2 thereof is closed. This allows, according to some embodiments, flow of the acid or base through the liquid feed outlet line 131, the first junction 138, the circulation line 135, the second junction 139 and the product line 150 without entering the bioreactor 130. As a result, the enzymatically active composite system 140 does not come in contact with the acid/base, according to some embodiments.

According to some embodiments, during step (c1) the first port 139p1 of the second junction 139 is closed.

According to some embodiments, during step (c1) the second port 139p2 of the second junction 139 is open.

According to some embodiments, during step (c1) the third port 139p3 of the second junction 139 is open.

Thus, according to some embodiments, during step (c1) the second junction is in its third open state. This allows, according to some embodiments, flow of the acid or base through the liquid feed outlet line 131, the first junction 138, the circulation line 135, the second junction 139 and the product line 150 without entering the bioreactor 130. As a result, the enzymatically active composite system 140 does not come in contact with the acid/base, according to some embodiments.

According to some embodiments, step (c1) further comprises flowing the basic and/or acidic solution or flowing both consecutively, through the product line 150 to the product container 180, thereby disinfecting the product container 180. It is to be understood that in such option, the liquid product composition has been evacuated from the product container 180 before flowing the basic and/or acidic solution(s) thereto.

According to some embodiments, step (c1) further comprises flowing the basic and/or acidic solution or flowing both consecutively, through the product line 150 to a separate container(s) dedicated to collecting bases and/or acids.

According to some embodiments, step (c1) further includes flowing the acidic/basic solution from the liquid feed 110 to the liquid feed outlet line 131.

Specific reference is now made to step (c2) of the present method. According to some embodiments, the present method includes step (c2) of contacting the enzymatically active composite system within the bioreactor 130 with an aqueous disinfecting composition, thereby producing a disinfected enzymatically active composite composition dispersed in the aqueous disinfecting composition. According to some embodiments, step (c2) further includes separating the disinfected enzymatically active composite system from the aqueous disinfecting composition, thereby producing a separated disinfected enzymatically active composite system.

According to some embodiments, the aqueous disinfecting composition comprises a quaternary ammonium compound.

According to some embodiments, the enzymatic reaction assembly 100 is structured to enable performing the disinfection according to steps (c1) and (c2) as detailed herein.

Step (c2) comprises contacting the enzymatically active composite system 140 within the bioreactor 130 with an aqueous disinfecting composition, thereby producing a disinfected enzymatically active composite composition dispersed in the aqueous disinfecting composition. According to some embodiments, step (c2) further comprises and separating the disinfected enzymatically active composite system 140 from the aqueous disinfecting composition, thereby producing a separated disinfected enzymatically active composite system.

According to some embodiments, washing a portion of the assembly, which does not include the bioreactor as described herein above, producing a disinfected enzymatically active composite composition dispersed in the aqueous disinfecting composition and producing a separated disinfected enzymatically active composite system, is performed at any order.

According to some embodiments, the aqueous disinfecting composition is a food grade composition.

According to some embodiments, the quaternary ammonium compound is represented by Formula (I):

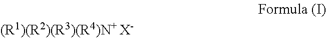

wherein
each one of R¹ and R² is independently a methyl;
each one of R³ and R⁴ is independently a C₂₋₁₆ alkyl; and
X is a halide.

According to some embodiments, X is selected from the group consisting of: chloride, bromide, and fluoride. According to some embodiments, X is chloride.

According to some embodiments, R³ is a C₄₋₁₄ alkyl. According to some embodiments, R³ is a C₆₋₁₂ alkyl. According to some embodiments, R³ is a C₈₋₁₂ alkyl. According to some embodiments, R³ is an unsubstituted alkyl. According to some embodiments, R³ is straight-chain alkyl.

According to some embodiments, R⁴ is a C₄₋₁₄ alkyl. According to some embodiments, R⁴ is a C₆₋₁₂ alkyl. According to some embodiments, R⁴ is a C₈₋₁₂ alkyl. According to some embodiments, R⁴ is an unsubstituted alkyl. According to some embodiments, R⁴ is straight-chain alkyl.

According to some embodiments, the quaternary ammonium compound is didecyldimethylammonium chloride.

According to some embodiments, the aqueous disinfecting composition of step (c2) further comprises a potassium salt, a calcium salt, or a combination thereof. According to some embodiments, the potassium salt in the aqueous disinfecting composition of step (c2) is at a concentration of at least 0.06M. According to some embodiments, the aqueous disinfecting composition of step (c2) further comprises a potassium salt, in a concentration of at least 0.01M. According to some embodiments, the aqueous disinfecting composition of step (c2) further comprises a potassium salt, in concentration in the range of 0.04M to 0.4M. According to some embodiments, the aqueous disinfecting composition of step (c2) is an aqueous disinfecting solution.

According to some embodiments, the enzymatically active agent has a pre-disinfected enzymatic activity in step (b) and a post-disinfected activity upon completion of step (c), wherein the post-disinfected activity is at least 50%, at least 55%, at least 60%, at least 65%, at least 70% the pre-disinfected enzymatic activity. Each possibility represents a separate embodiment of the invention.

The term “enzymatic activity”, sometimes also referred to as “catalytic activity” or “catalytic efficiency”, is generally known to the person skilled in the art and refers to the conversion rate of an enzyme and is usually expressed by means of the ratio kₖₐₜ/K_{M}, wherein kₖₐₜ is the catalytic constant (also referred to as turnover number) and the K_{M} value corresponds to the substrate concentration, at which the reaction rate lies at half its maximum value.

According to some embodiments, the enzymatically active composite system of step (b) has an initial microbial count, and wherein step (c2) entails disinfecting said enzymatically active composite system 140, wherein the separated disinfected enzymatically active composite system 140 has a final microbial count. according to some embodiments, the initial microbial count is higher than the final microbial count. As used herein the microbial count is measured by counting colony forming units (CFU). It is to be understood that non-proliferating microbial cells, do not form any colonies, hence their presence as enzymatically active agent active does not affect the microbial count. According to some embodiments, the initial microbial count is at least 10⁻³ higher than the final microbial count. According to some embodiments, there is a reduction of at least 10⁻³ in the microbial count (e.g., CFU) of the initial microbial count following disinfecting according to step (c).

According to some embodiments, the enzymatically active composite system of step (b) has an initial microbial count, and wherein step (c2) entails disinfecting said enzymatically active composite system 140, wherein the separated disinfected enzymatically active composite system 140 has a final microbial count. according to some embodiments, the initial microbial count is higher than the final microbial count. As used herein the microbial count is measured by counting colony forming units (CFU). It is to be understood that non-proliferating microbial cells, do not form any colonies, hence their presence as enzymatically active agent active does not affect the microbial count. According to some embodiments, the initial microbial count is at least 103 higher than the final microbial count. According to some embodiments, there is a reduction of at least 10⁻³ in the microbial count (e.g., CFU) of the initial microbial count following disinfecting according to step (c).

According to some embodiments, step (c2) comprises maintaining the enzymatically active composite system with the aqueous disinfecting composition in the bioreactor at a temperature in the range of 4° C. to 40° C., including each value and sub-range within the specified range.

Reference is now made to the method of the present invention as a whole. According to some embodiments, the method comprises repeating one or more times the enzymatic reaction according to (b) after the disinfection of (c).

According to some embodiments, the method comprises repeating a plurality of times the enzymatic reaction according to (b) after the disinfection of (c). According to some embodiments, the plurality of times includes at least 2, at least 5, at least 10, or at least 20. Each possibility represents a separate embodiment of the invention.

According to some embodiments, the one or more times of the enzymatic reaction according to (b) after the disinfection of (c) comprises a final repeat, wherein the time from an initial performance of the enzymatic reaction according to (b) to the final repeat is at least 30 days, at least 45 days, at least 60 days, or at least 90. Each possibility represents a separate embodiment of the invention. According to some embodiments, during said days the enzymatically active composite system 140 is maintained within the reactor 130. According to some embodiments, during said days no additional composite is added. According to some embodiments, the one or more times of the enzymatic reaction according to (b) after the disinfection of (c) comprises a final repeat, is a semi-continuous process, which does not include opening the bioreactor 130 for replacement of the enzymatically active composite system 140.

Reference is now made to utilization of the present method in the food and beverage industry for reducing the content of mono- and/or disaccharide in fruit and/or vegetable juices.

According to some embodiments, the present method is for reducing the monosaccharide and/or disaccharide content of a fruit and/or vegetable juice. According to some embodiments, the present method is for reducing the monosaccharide content of a fruit and/or vegetable juice. According to some embodiments, the present method is for reducing the disaccharide content of a fruit and/or vegetable juice.

The term “fruit and/or vegetable juice” refer to any juice obtain from any fruit and/or vegetable, including mixtures of different fruits and/or vegetables.

According to some embodiments, the starting liquid composition comprises an initial fruit or vegetable juice.

As used herein, the terms “initial fruit or vegetable juice”, “initial juice”, “starting juice” or “starting juice product” which may be used interchangeably, refer to the juice before being subjected to the enzymatic reaction assembly of the present invention. The initial juice may be, but not limited to, a natural juice freshly squeezed, or extracted otherwise from a fruit or vegetable, or a juice after preliminary processing step(s), such as pulp removal, filtration(s), titrations, heat treatment(s) (e.g., pasteurization) and the like. According to some embodiments, the starting liquid composition is initial fruit or vegetable juice.

According to some embodiments, the juice is obtained from at least one type of fruit, vegetable, or any combination thereof. Any method as is known in the art for forcing the juice out of the source material and optionally for pre-treating the obtained natural juice before it is contacted with the enzymatically active resin of the present invention can be used with the teachings of the present invention. According to some embodiments, the term “juice” encompasses any one of freshly squeezed juice, clear juice, nectar, juice concentrate, fruit drink, smoothie, puree, or any combination thereof. Each possibility represents a separate embodiment of the present invention.

Any fruit or vegetable juice containing sugar can be subjected to the method according to the teachings of the present invention. Thus, the process is equally applicable to apple, cranberry, pear, peach, plum, apricot, nectarine, grape, cherry, currant, raspberry, gooseberry, blackberry, blueberry, strawberry, lemon, orange, grapefruit, potato, tomato, celery, rhubarb, carrot, beet, cucumber, pineapple, custard-apple, coconut, pomegranate, kiwi, mango, Papaya, banana, watermelon, guava, passion fruit, cantaloupe, and combinations thereof. According to some embodiments, the juice is of apple, pear, strawberry, orange, pineapple, grape or cherry. Each possibility represents a separate embodiment of the present invention.

According to some embodiments, fruit or vegetable juice is selected from a group consisting of orange juice, lemon juice, grapefruit juice, grape juice, apple juice, pear juice, cherry juice, cranberry juice, mango juice, strawberry juice, pineapple juice, guava juice, peach juice, plum juice, apricot juice, nectarine juice, current juice, raspberry juice, gooseberry juice, blackberry juice, blueberry juice, pomegranate juice, kiwi juice, banana juice, Papaya juice, watermelon juice, cantaloupe juice, coconut juice, passion fruit juice, beetroot juice, tomato juice, celery juice, rhubarb juice, carrot juice and mixtures thereof. Each possibility represents a separate embodiment of the invention.

According to some embodiments, the fruit or vegetable juice is selected from a group consisting of orange juice, grape juice, apple juice, pear juice, cherry juice, strawberry juice, and pineapple juice.

According to some embodiments, the initial fruit or vegetable juice comprises an initial total monosaccharide and disaccharide concentration.

The term(s) “mono and/or disaccharide” as used herein refers to sugars/carbohydrates containing either one or two sugar/saccharide unit(s), respectively, and which naturally present within a vegetable or fruit juice. According to some embodiments monosaccharide or disaccharide, comprise Sucrose, Glucose, and Fructose (SGF). Accordingly, as used herein, the terms “reducing monosaccharide or disaccharide concentration”, “reducing monosaccharide or disaccharide content”, “reducing sugar concentration” “reducing sugar content”, “reduced monosaccharide or disaccharide concentration” or “reduced monosaccharide or disaccharide content” refers to the reduction of mono- and/or disaccharides, particularly glucose, fructose, sucrose, or any combination thereof.

The terms “total sugar content”, “total sugar concentration”, “total mono or mono and/or disaccharide content”, “total mono or mono and/or disaccharide concentration” refer to the combination of sucrose content, glucose content and fructose content in a juice, unless specifically specified otherwise. The total sugar content ranges between 4% to 20% sugar weight per juice volume (w/v). It is to be understood that when referring to a percentage of the sucrose content, or glucose content, or fructose content out of the total sugar content as described hereinbelow, it is meant, for example, 10% fructose out of a total sugar content of 20% in the natural juice, that is 2% w/v out of the total volume of the juice.

As used herein the term “initial total monosaccharide and disaccharide concentration”, “initial total monosaccharide and disaccharide content”, “initial total sugar concentration” or “initial sugar content”, which may be used interchangeably, refers to the sugar/monosaccharide and disaccharide content/concentration of initial fruit or vegetable juice/initial juice/starting juice/starting juice product.

According to some embodiments, the initial total monosaccharide and disaccharide concentration is in the range of 2% to 30% w/v, including each value and sub-range within the specified range.

According to some embodiments, the initial fruit or vegetable juice comprises an initial glucose concentration. According to some embodiments, the initial glucose concentration is in the range of 1% to 20%, including each value and sub-range within the specified range.

According to some embodiments, the initial fruit or vegetable juice comprises an initial fructose concentration. According to some embodiments, the initial fructose concentration is in the range of 1% to 20%, including each value and sub-range within the specified range.

According to some embodiments, the initial fruit or vegetable juice comprises an initial sucrose concentration. According to some embodiments, the initial sucrose concentration is in the range of 1% to 10%, including each value and sub-range within the specified range.

According to certain embodiments, the sugar composition of the initial juice comprises from about 10% to about 90% fructose out of the total sugar content. According to certain embodiments, the sugar composition of the initial juice comprises from about 10%, about 20%, about 30% or about 40% to about 60%, about 70%, about 80% or about 90% fructose out of the total sugar content.

According to certain embodiments, the sugar composition of the initial juice comprises from about 10% to about 70% glucose out of the total sugar content. According to certain embodiments, the sugar composition of the initial juice comprises from about 10%, about 20%, about 30% or about 40% to about 60%, about 70%, about 80% or about 90% glucose out of the total sugar content.

According to certain embodiments, the sugar composition of the initial juice comprises from about 1% to about 70% sucrose out of the total sugar content. According to certain embodiments, the sugar composition of the sugar containing beverage comprises from about 1%, about 20%, about 30% or about 40% to about 60%, about 70%, about 80% or about 90% sucrose out of the total sugar content.

According to some embodiments, the stating liquid of step (b1) comprises at least a portion of the initial fruit or vegetable juice.

According to some embodiments, step (b2) comprises contacting the initial fruit or vegetable juice with the enzymatically active composite system 140 in the bioreactor 130.

According to some embodiments, the contacting the initial fruit or vegetable juice with the enzymatically active composite system 140 induces an enzymatic reaction.

According to some embodiments, the enzymatic reaction catalyzes a transformation of monosaccharides, disaccharides, or both into at least one of: at least one sugar alcohol; at least one a sugar acid; at least one oligosaccharide; at least one polysaccharide; or any combination thereof.

According to some embodiments, the enzymatic reaction catalyzes a transformation of monosaccharides to sugar acids. According to some embodiments, the enzymatic reaction catalyzes a transformation of monosaccharides to sugar alcohols. According to some embodiments, the enzymatic reaction catalyzes a transformation of monosaccharides and/or disaccharides into at least one oligosaccharide; at least one polysaccharide; or any combination thereof.

The term “sugar alcohol” as used herein refers to a chemical derivative derived from monosaccharides by the reduction of the aldehyde or ketone group to an alcohol group using, inter alia, glucose fructose oxidoreductase (GFOR-EC number 1.1.99.28) enzymes. Sugar alcohols are slowly and incompletely absorbed from the small intestine into the blood, consequently contribute fewer calories. Once absorbed they are converted to energy by processes that require little or no insulin. According to certain exemplary embodiments, the sugar alcohol is sorbitol.

According to some embodiments, the sugar acid comprises gluconic acid.

The term “gluconic acid” as used herein refers to the C1-oxidized form of D-glucose, where the aldehyde group has become oxidized using GFOR to the corresponding carboxylic acid. Gluconic acid is known in the food industry as an acidity regulator, thus may contribute to the stability of a food product. Furthermore, gluconic acid does not add any calories to the obtained food product (its calorie value is 0), and it may serve as a carrier for iron, calcium and other ions, based on its capability to form gluconate salt with such ions, which may be present in the food product. The gluconate salts provide for better bioavailability of these essential microelements.

According to some embodiments, the enzymatically active agent comprises a plurality of non-proliferating bacterial cells, comprising glucose fructose oxidase reductase (GFOR), glucose oxidase (GOX) or sucrose fructosyltransferase (SFTase). Each possibility represents a separate embodiment of the invention.

According to some embodiments, step (b2) comprises contacting the initial fruit or vegetable juice with the enzymatically active composite system in the bioreactor, thereby inducing an enzymatic reaction, which catalyze a transformation of glucose, fructose, sucrose or any combination thereof into at least one of sorbitol, gluconic acid, at least one fructooligosaccharides, and any combination thereof into at least one of sorbitol, gluconic acid, at least one fructooligosaccharides, and any combination thereof.

According to some embodiments, the enzymatic reaction produces a processed juice. According to some embodiments, the processed juice a final total monosaccharide and disaccharide concentration.

The terms “treated juice”, “final juice”, or “processed juice” which may be used interchangeably, refer to the juice product obtained after being subjected to the enzymatic reaction assembly of the present invention. According to some embodiments, the treated juice comprises reduced content of at least glucose and/or fructose and/or sucrose compared to the content present in the starting juice, and optionally elevated content of sugar alcohol (e.g., sorbitol), and/or gluconic acid, and/or fructooligosaccharides (FOS). According to some embodiments, the liquid product composition is a final juice.

As used herein the term “final total monosaccharide and disaccharide concentration”, “final total monosaccharide and disaccharide content”, “final total sugar concentration” or “final sugar content”, which may be used interchangeably, refers to the sugar/monosaccharide and disaccharide content/concentration of a final/treated/processed juice.

According to some embodiments, the initial total monosaccharide and disaccharide concentration is higher than the final total monosaccharide and disaccharide concentration. According to some embodiments, the initial total monosaccharide and disaccharide concentration is at least 25% higher than the final total monosaccharide and disaccharide concentration. According to some embodiments, the initial total monosaccharide and disaccharide concentration is at least 50% higher than the final total monosaccharide and disaccharide concentration. According to some embodiments, the initial total monosaccharide and disaccharide concentration is at least 100% higher than the final total monosaccharide and disaccharide concentration.

According to some embodiments, the final total monosaccharide and disaccharide concentration is at least 10%, at least 25%, at least 50% or at least 75% lower than the initial total monosaccharide and disaccharide concentration. Each possibility represents a separate embodiment of the invention.

According to some embodiments, the final total monosaccharide and disaccharide concentration is 10% to 75% lower than the initial total monosaccharide and disaccharide concentration, including each value and sub-range within the specified range.

According to certain embodiment, the total sucrose+glucose+fructose (SGF) content in the treated juice obtained by the process of the present invention is reduced by at least 5%, 10%, 15%, 20%, 25%, 30%, 35%, 40%, 45%, 50%, 55%, 60%, 65%, 70%, 75%, 80%, 85%, 90%, and 95% or more compared to the total SGF content in the starting juice. Each possibility represents a separate embodiment of the invention.

According to some embodiments, the starting juice comprises fructose, the fructose content in the treated juice is reduced by at least 5%, 10%, 15%, 20%, 25%, 30%, 35%, 40%, 45%, 50%, 55%, 60%, 65%, 70%, 75%, 80%, 85%, 90%, and 95% or more compared to its content in said starting juice.

According to some embodiments, the starting juice comprises sucrose, the sucrose content in the treated juice is reduced by at least 5%, 10%, 15%, 20%, 25%, 30%, 35%, 40%, 45%, 50%, 55%, 60%, 65%, 70%, 75%, 80%, 85%, 90%, and 95% or more compared to its content in said starting juice.

According to some embodiments, the starting juice comprises glucose, the glucose content in the treated juice is reduced by at least 5%, 10%, 15%, 20%, 25%, 30%, 35%, 40%, 45%, 50%, 55%, 60%, 65%, 70%, 75%, 80%, 85%, 90%, and 95% or more compared to its content in the starting food product.

According to some embodiments, the treated juice of the present invention may have a calorie content of less than 50%, 45%, 40%, 35%, 30%, 25%, 20%, 15%, 10% or less of the starting juice from which it is derived. At the same time, the reduced calorie treated juice may have a flavor profile and mouth feel which are comparable to the starting juice.

According to some embodiments, the initial juice comprises no oligosaccharide and polysaccharide or has an initial total oligosaccharide and polysaccharide concentration and the processed juice has a final total oligosaccharide and polysaccharide concentration, which is higher that the initial total oligosaccharide and polysaccharide concentration.

According to some embodiments, the final total oligosaccharide and polysaccharide concentration is at least 50% higher that the initial total oligosaccharide and polysaccharide concentration. According to some embodiments, the final total oligosaccharide and polysaccharide concentration is at least 100% higher that the initial total oligosaccharide and polysaccharide concentration.

According to some embodiments, the final total oligosaccharide and polysaccharide concentration is at least 5%, 10%, 15%, 20%, 25%, 30%, 35%, 40%, 45%, 50%, 55%, 60%, 65%, 70%, 75%, 80%, 85%, 90%, and 95% higher that the initial total oligosaccharide and polysaccharide concentration.

According g to some embodiment, the total oligosaccharide and polysaccharide content in the treated juice obtained by the process of the present invention is elevated by at least 5%, 10%, 15%, 20%, 25%, 30%, 35%, 40%, 45%, 50%, 55%, 60%, 65%, 70%, 75%, 80%, 85%, 90%, and 95% or more compared to the total SGF content in the starting juice.

According to some embodiments, the FOS content in the treated juice is elevated by at least 5%, 10%, 15%, 20%, 25%, 30%, 35%, 40%, 45%, 50%, 55%, 60%, 65%, 70%, 75%, 80%, 85%, 90%, and 95% or more compared to its content in the starting juice.

According to some embodiments, the dietary fibers content in the treated juice is elevated by at least 5%, 10%, 15%, 20%, 25%, 30%, 35%, 40%, 45%, 50%, 55%, 60%, 65%, 70%, 75%, 80%, 85%, 90%, and 95% or more compared to its content in the starting juice.

According to some embodiments, the initial juice does not comprise detectable amount of sorbitol and the treated juice comprises detectable amounts of sorbitol. According to certain embodiments, the sorbitol content in the treated juice is elevated by at least 5%, 10%, 15%, 20%, 25%, 30%, 35%, 40%, 45%, 50%, 55%, 60%, 65%, 70%, 75%, 80%, 85%, 90%, and 95% or more compared to its content in the starting juice.

According to some embodiments, the processed juice has higher content of dietary fiber compared to the initial fruit or vegetable juice. According to some embodiments, the dietary fiber comprises fructan(s). According to some embodiments, the dietary fiber comprises fructooligosaccharide (FOS).

As used herein the term “dietary fiber” refers to a complex dietary component, including carbohydrate polymers and oligomers, which makes up the non-digestible components of food, particularly fruit, vegetable and grains. All dietary fibers resist digestion in the small intestine and pass into the large intestine intact but differ in their physiochemical characteristics (e.g., solubility, viscosity, and fermentability), which determine their functionality in the gut and to what degree they are accessible by microbes. Most soluble fibers can be fermented by the gut microbiota, partially or completely, dependent on their chemical structure. Dietary fibers can be defined on the basis of their chemical compounds, on the basis of their functional compounds, or both. Slight differences in definitions of dietary fibers exist due to the wide range of non-digestible fibers that occur in nature. The European Food Safety Authority (EFSA) defines dietary fiber as “non-digestible carbohydrates plus lignin.” These include non-starch polysaccharides (NSP) cellulose, hemicelluloses, pectins, hydrocolloids (i.e., gums, mucilages, and β-glucans), resistant oligosaccharides, resistant starch (consisting of physically enclosed starch, some types of raw starch granules, retrograded amylose, chemically and/or physically modified starches), and lignin associated with the dietary fiber polysaccharides. According to certain exemplary embodiments, the term “dietary fibers” is used herein to refer to soluble fibers.

Fructans are fructose polymers produced by plants, yeasts, fungi, and bacteria and are considered dietary fibers. Fructans with a short chain length are known as fructooligosaccharides (FOS), whereas longer chain fructans are termed inulins or levans. FOS can be obtained by the transfructosylation action of the enzyme fructosyltransferase (FTase) on sucrose, sucrose fructosyltransferase (SFTase-EC number 2.4.1.99. FOS are composed of linear chains of fructose units, linked by beta (2-1) bonds, having the general formula of Glucose-Fructosen (GFn). The number of fructose units ranges from 2 to 60 and often initiate with a glucose unit. Dietary FOS are not hydrolyzed by small intestinal glycosidases and reach the cecum structurally unchanged. There, they are metabolized by the intestinal microflora to form short-chain carboxylic acids, L-lactate, CO₂, hydrogen and other metabolites. FOS have a number of interesting properties, including a low sweetness intensity; they are also calorie free, non-cariogenic and are considered as soluble dietary fiber. Furthermore, FOS have important beneficial physiological effects such as a prebiotic effect, improved mineral absorption and decreased levels of serum cholesterol, triacylglycerols and phospholipids. FOS stimulate the growth of nonpathogenic intestinal microflora and increases fecal bolus and the frequency of defection (Sabater-Molina M, Larqué E, Torrella F, Zamora S. Dietary fructooligosaccharides and potential benefits on health. J Physiol Biochem. 2009 September; 65 (3): 315-28). FOSs are naturally produced by a large variety of microorganisms including, inter alia, Aspergillus japonicus (AJ).

According to some embodiments, the method of reducing the content of mono- and/or disaccharide in fruit and/or vegetable juices comprises repeating one or more times the enzymatic reaction according to (b) after the disinfection of (c). According to some embodiments, the one or more times of the enzymatic reaction according to (b) after the disinfection of (c) is a semi-continuous process which comprises a final repeat, wherein the time from an initial performance of the enzymatic reaction according to (b) to the final repeat is at least 30 days, at least 45 days, at least 60 days, or at least 90 days which does not include opening the bioreactor 130 for replacement of the enzymatically active composite system 140, which remains in the bioreactor 130 during the semi-continuous process.

According to some embodiments, the final juice produced in step (b) after the final repeat and after arriving to the product container 180 of the enzymatic reaction assembly 100 has microbial counts (namely, total microbial count and strain specific microbial count), denoted herein “final juice microbial count”. It is to be noted that the final juice produced in step (b) after the final repeat may be obtained upon repeating one or more times the enzymatic reaction according to step (b) after the disinfection of step (c). According to some embodiments, the initial juice before being loaded to the liquid feed 110 of the enzymatic reaction assembly 100 has microbial counts, denoted herein as “initial juice microbial count”.

According to some embodiments, the total microbial count of the processed juice produced in step (b) of the final repeat, final juice microbial count, is no more than 10 CFU/ml, 50 CFU/ml, 100 CFU/ml, 200 CFU/ml, 500 CFU/ml, 750 CFU/ml, or 1000 CFU/ml. Each possibility represents a separate embodiment of the invention.

According to some embodiments, the final juice microbial count is no more than 1×10⁻² CFU/gr of total aerobic microorganisms, no more than 10 CFU/gr molds, no more than 1×10⁻² CFU/gr yeasts, no more than 10 CFU/gr E. coli 0157, no detectable bacteria from the Salmonella genus, and no detectable of Listeria monocytogenes.

According to some embodiments, the final juice microbial count is no more than 1×10⁻² CFU/gr of total aerobic microorganisms. According to some embodiments, the final juice microbial count is no more than 1×10⁻² CFU/gr yeasts. According to some embodiments, the final juice microbial count is no more than 10 CFU/gr E. coli 0157. According to some embodiments the final juice comprises no detectable bacteria from the Salmonella genus. According to some embodiments the final juice comprises no detectable Listeria monocytogenes.

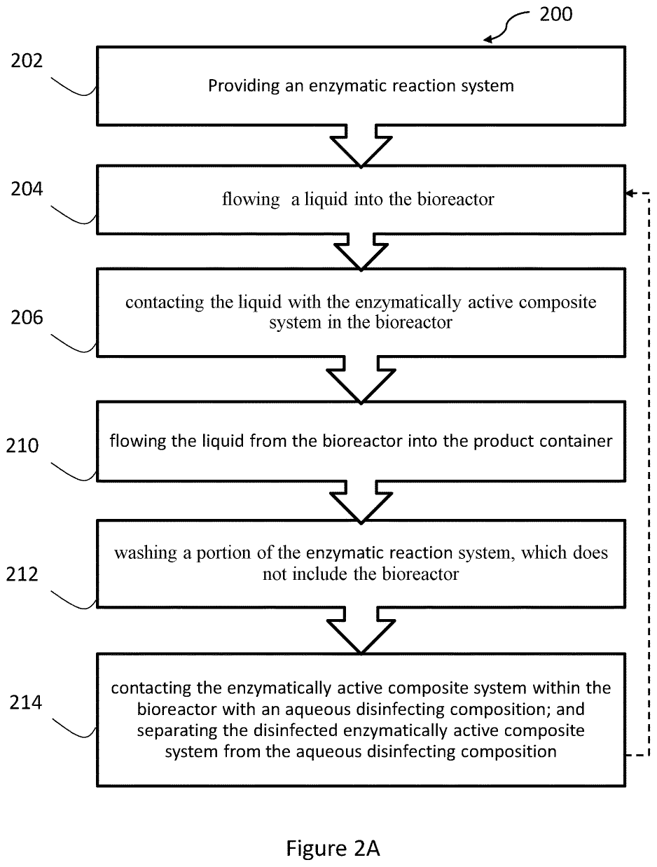

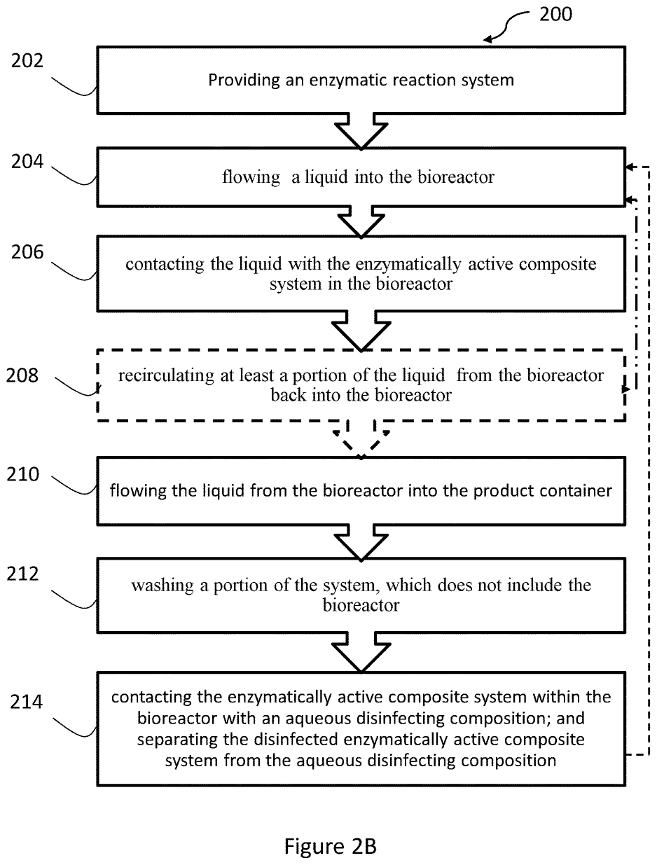

According to some embodiments a, microbial count of a final juice obtained in a step (b4) which is directly following step (c) is lower than a microbial count of a final juice obtained in step (b4) which is directly before said step (c). FIG. 2A and FIG. 2B are flowcharts that visually illustrate specific methods of the present invention. As can be seen in FIG. 2A and FIG. 2B: step 202 refers to step (a) as described herein, according to some embodiments; step 204 refers to step (b1) as described herein, according to some embodiments; step 206 refers to step (b2) as described herein, according to some embodiments; step 208 refers to step (b3) as described herein, according to some embodiments; step 210 refers to step (b4) as described herein, according to some embodiments; step 212 refers to step (c1) as described herein, according to some embodiments; step 214 refers to step (c2) as described herein, according to some embodiments. Therefore, as can be appreciated by the person having ordinary skill in the art each embodiment relating to steps 202, 204, 206, 208, 210, 212, and 214 similarly relates to steps (b1), (b2), (b3), (b4), (c1), and (c2), respectively. It is further to be understood that broken lines and arrows (e.g., step 210 in FIG. 2B, the arrows between steps 214 and 208 to step 204) represents optional steps according to the methods FIGS. 2A and 2B.

### EXAMPLES

### Example 1: Disinfection Procedure of an Enzymatically Active Composite System and Enzymatic Reaction Assembly

To evaluate the effectivity of the in-situ disinfection method of the present invention, microbial counts of two batches of initial apple juice and treated juice were compared over 33 or 35 days, respectively, of operation of an enzymatic reaction. The operation of the enzymatic reaction within the enzymatic reaction assembly included the disinfection steps according to the teaching of the present invention, and was performed without the replacement of the enzymatically active composite system (i.e., beads). To that end, a total of 10 Kg of a composite system according to the present invention was loaded into a bioreactor of an enzymatic reaction assembly. The composite system used in the present experiment comprised non-proliferating Zymomonas mobilis and Aspargillus japonicus immobilized to carrageenan beads.

Next, a clear commercially-available apple juice having ~12.0° Bx was loaded into the liquid feed and fed into the enzymatic reaction assembly, particularly into the bioreactor at a flow rate of 0.15-1 L/hr. The juice was tittered to pH~6 using aqueous KOH 50% w/w. The juice was continuously fed into the bioreactor for 60 consecutive days, namely Day 1 to Day 60. During these operation days, an enzymatic reaction of lowering the mono/disaccharides content took place.

Downtimes were executed on vacations as indicated in Tables 1 and 2, i.e., in days 4-6 and 13-15 for 72 hrs each time. During downtimes, the enzymatically active composite system was contained together with 20 L juice within the bioreactor, and the juice flow into and out from the bioreactor was ceased. The bioreactor, which contained the enzymatically active composite system therein, was disconnected from the other parts of the enzymatically reaction assembly. As a result, the bioreactor was no longer in fluid communication with any other parts of the assembly. Consecutively, liquids could be loaded into the assembly and flown through the assembly parts while bypassing the bioreactor, thereby washing a part of the assembly, which does not include the bioreactor. During downtimes, methyl paraben was added to the composite system, which was kept at pH~6 and stirred within the bioreactor at 4° C.

Following downtime, continuous flow of juice into and out from the bioreactor was renewed. The first 60 L of juice arriving at the product container following downtime was discarded.

Disinfection procedures of the present invention were performed as indicated in Tables 1-2, for a duration of 3 hrs. During the disinfection of the enzymatic reaction assembly, similarly to downtimes, the enzymatically active composite system and the juice was contained within the bioreactor, and the juice flow into and out from the bioreactor ceased. The bioreactor was disconnected from the other parts of the system. Disinfection of both the bioreactor part and the other parts of the assembly, which does not include the bioreactor, was conducted simultaneously.

The disinfection of the bioreactor (and the enzymatically active composite system therein), included the addition of an aqueous solution of didecyldimethylammonium chloride (400 ppmM). In addition, KCl (0.3M), was added to the bioreactor.

The disinfection of the other parts of the assembly (other than the bioreactor) included washing with (i) 60 L aqueous NaOH at 80° C.; (ii) water washing until pH below 8 was reached (iii); 60 L HCl (1%) at 80° C. (vi) water washing until pH above 6 was reached.

Following the simultaneous yet distinct disinfection procedures of the bioreactor and the other assembly parts, the entire enzymatic reaction assembly was washed with 60 L of the commercial juice. The washing juice was then discarded.

Additional initial juice was then fed to the assembly, and an additional round of the continuous flow of juice into and out from the bioreactor was renewed.

During the entire period of operation, samples of initial juice and final juice (following treatment) were collected. Aliquots of the samples streaked onto Standard methods Agar (SMA) Total count plates and Oxytetracycline Glucose Yeast extract (OGY) for yeast and molds plates and incubated for 72 hr at 30° C. Following the incubation, total microbial counts (SPC) were measured. The microbial counts in the juice are summarized in Tables 1-2. In addition, the ability of the system to reduce the sugar content throughout the procedure was monitored periodically, as indicated in Tables 1-2 (wherein “SGF” represents the total content of sucrose, glucose, and fructose).

TABLE 1
microbial count and sugar %

| | Flow | | SPC |
|---|---|---|---|
| Day | L/hr | SGF % | CFU/ml |
| 1 | 1 | 7.1 | |
| 2 | 0.155 | 19.4 | <10 |
| 2 | 0.6 | 9.8 | |
| 3 | 0.23 | 14.1 | <10 |
| 3 | 0.367308 | 11.8 | |
| 4 | Downtime | | |
| 5 | Downtime | | |
| 6 | Downtime | | |
| 10 | Disinfection procedure | | |
| 11 | 0.63 | 10.7 | <10 |
| 12 | 0.16 | 17.1 | |
| 12 | 0.36 | 11.4 | |
| 13 | Downtime | | |
| 15 | Downtime | | |
| 16 | Downtime | | |
| 17 | Disinfection procedure | | |
| 18 | 0.629643 | 10.1 | <10 |
| 19 | 0.162857 | 14.8 | <10 |
| 19 | 0.379032 | 9.3 | |
| 22 | Disinfection procedure | | |
| 23 | 0.273333 | 10.8 | <10 |
| 23 | 0.3875 | 11.7 | |
| 24 | 0.265846 | 14.2 | 4000 |
| 24 | 0.630571 | 9.1 | |
| 25 | 0.165 | 16.8 | 1000000 |
| 26 | 0.163077 | 16.3 | 560 |
| 26 | 0.369495 | 11.8 | |
| 22 | Disinfection procedure | | |
| 30 | 0.27 | 11.0 | <10 |
| 30 | 0.655294 | 10.4 | |
| 31 | 0.255455 | 12.8 | <10 |
| 31 | 0.391318 | 12.0 | |
| 32 | 0.16875 | 15.9 | <10 |
| 32 | 0.38 | 11.4 | |
| 33 | 0.234 | 14.3 | 1300 |
| 33 | 0.609145 | 8.4 | |

TABLE 2
microbial count and sugar reduction

| | % Sugar | SPC |
|---|---|---|
| Day | Reduction | CFU/ml |
| 2 | 35% | 50 |
| 4 | 22% | 1000000 |
| 4 | 16% | 1000000 |

| | |
|---|---|
| 5 | Downtime |
| 6 | Downtime |
| 7 | Downtime |
| 8 | Disinfection procedure |

| | | |
|---|---|---|
| 9 | 18% | <10 |
| 10 | 47% | <10 |
| 12 | 30% | <10 |
| 13 | 39% | <10 |

| | |
|---|---|
| 14 | Downtime |
| 15 | Downtime |
| 16 | Disinfection procedure |

| | | |
|---|---|---|
| 17 | 30% | <10 |
| 21 | 14% | <10 |

| | |
|---|---|
| 25 | Disinfection procedure |

| | | |
|---|---|---|
| 26 | 31% | 10 |
| 27 | 18% | 180 |
| 27 | 32% | 120 |
| 28 | | 30 |

| | |
|---|---|
| 27 | Downtime |
| 28 | Downtime |
| 29 | Downtime |
| 30 | Disinfection procedure |

| | |
|---|---|
| 31 | 180 |
| 35 | 200 |

| | |
|---|---|
| 35 | Disinfection procedure |

| | |
|---|---|
| 35 | <10 |

In conclusion, the data presented so far demonstrates that the cleaning/sanitizing procedure of the present invention does not harm the enzymatic activity of the composite system, while maintaining a sufficiently disinfected product juice. Hence, the present procedure can be used for cleaning the composite and assembly periodically (e.g., twice a week) to maintain sufficiently low microorganism count in the final juice.

1. A method of performing an enzymatic reaction and disinfecting an enzymatically active composite system, the method comprising:
(a) providing an enzymatic reaction assembly, comprising:
a liquid feed, which contains a starting liquid composition;
a bioreactor, which contains an enzymatically active composite system, the composite system comprising an enzymatically active agent immobilized to a support;
a liquid feed outlet line, which extends from the liquid feed to a first junction, wherein the first junction comprises a first port connected to the feed outlet line, a second port connected to a bioreactor inlet line and a third port connected to a circulation line; wherein the bioreactor inlet line is in fluid communication with the bioreactor and the circulation line is in fluid communication with a second junction;
a bioreactor outlet line, which extends from the bioreactor to the second junction, wherein the second junction comprises a first port connected to the bioreactor outlet line, a second port connected to the circulation line and a third port connected to a product line; and
a product container, which is in fluid communication with the product line;
(b) performing an enzymatic reaction, comprising:
(b1) flowing the starting liquid composition from the liquid feed through the liquid feed outlet line, the first junction and the bioreactor inlet line into the bioreactor;
(b2) contacting the starting liquid composition with the enzymatically active composite system in the bioreactor, thereby inducing an enzymatic reaction, to produce a liquid product composition;
(b3) recirculating the liquid product composition from the bioreactor through the bioreactor outlet line, the second junction, the circulation line, the first junction and the bioreactor inlet line, back into the bioreactor, wherein step (b3) is optional and may be performed one or more times;
(b4) flowing the liquid product composition from the bioreactor through the bioreactor outlet line, the second junction and the product line into the product container;
(c) performing a disinfection, comprising performing steps (c1) and (c2) at any order, wherein
step (c1) comprises flowing an acidic solution, flowing a basic solution or flowing both consecutively, through the liquid feed outlet line, the first junction, the circulation line, the second junction and the product line, thereby washing a portion of the assembly, which does not include the bioreactor; and
step (c2) comprises contacting the enzymatically active composite system within the bioreactor with an aqueous disinfecting composition, thereby producing a disinfected enzymatically active composite composition dispersed in the aqueous disinfecting composition; and separating the disinfected enzymatically active composite system from the aqueous disinfecting composition, thereby producing a separated disinfected enzymatically active composite system, wherein the aqueous disinfecting composition comprises a quaternary ammonium compound;
wherein the enzymatically active composite system is contained within the bioreactor throughout steps (c1) and (c2);
wherein the method further comprises repeating one or more times the enzymatic reaction according to (b) after the disinfection of (c).

2.-45. (canceled)

46. The method of claim 1, wherein the one or more times of the enzymatic reaction according to (b) after the disinfection of (c) comprises a final repeat, wherein the time from an initial performance of the enzymatic reaction according to (b) to the final repeat is at least 30 days, during which the enzymatically active composite system is maintained within the reactor.

47. The method of claim 1, wherein the aqueous disinfecting composition is a food grade composition.

48. The method of claim 1, wherein the quaternary ammonium compound is didecyldimethylammonium chloride.

49. The method of claim 1, wherein the aqueous disinfecting composition of step (c2) further comprises a potassium salt, a calcium salt, or a combination thereof, at a concentration of at least 0.06M.

50. The method of claim 1, wherein the enzymatically active composite system comprises the enzymatically active agent bonded to the support, entrapped within the support, encapsulated within the support, embedded in the support or a combination thereof.

51. The method of claim 1, wherein the enzymatically active composite system is in the form of beads.

52. The method of claim 1, wherein the support is substantially water insoluble at 25° C. and comprises an anionic polymer comprising a polymeric backbone bonded to a plurality of negatively charged functional groups, selected from the group consisting of: carboxylate, sulfate, and sulfonate; and potassium countercations.

53. The method of claim 52, wherein the potassium countercations and the negatively charged functional groups are present at a ratio of at least 1:2.

54. The method of claim 52, wherein the support comprises a combination of alginate and gellan gum or a combination of carrageenan and xanthan gum.

55. The method of claim 1, wherein the enzymatically active agent comprises a plurality of non-proliferating bacterial cells, a plurality of non-proliferating fungal cells or both.

56. The method of claim 1, wherein the enzymatically active agent catalyzes a transformation of glucose, fructose, sucrose, or any combination thereof into at least one of sorbitol, gluconic acid, at least one fructooligosaccharides, and any combination thereof.

57. The method of claim 1, wherein the enzymatically active agent has a pre-disinfected enzymatic activity in step (b) and a post-disinfected activity upon completion of step (c), wherein the post-disinfected activity is at least 50% of the pre-disinfected enzymatic activity.

58. The method of claim 1, wherein step (c1) comprises washing the portion of the bioreaction assembly with an aqueous basic solution, wherein the basic solution comprises NaOH, KOH or both, wherein the basic solution has pH in the range of 11 to 13.

59. The method of claim 1, wherein washing the portion of the assembly with an aqueous basic solution is performed at a temperature in the range of 50° C. to 100° C.

60. The method of claim 1, wherein step (c1) comprises washing the portion of the assembly with an aqueous acidic solution, wherein the acidic solution has pH in the range of 2 to 4.

61. The method of claim 58, wherein step (c1) comprises
(ia) washing the portion of the assembly with the aqueous basic solution,
(iia) washing the portion of the assembly with a pH neutral aqueous solution, and
(iiia) washing the portion of the assembly with the aqueous acidic solution; or
(ib) washing the portion of the assembly with the aqueous acidic solution,
(iib) washing the portion of the assembly with a pH neutral aqueous solution, and
(iiib) washing the portion of the assembly with the aqueous basic solution.

62. The method of claim 1, for reducing the monosaccharide or disaccharide content of a fruit or vegetable juice,
wherein the starting liquid composition comprises an initial fruit or vegetable juice, which comprises an initial total monosaccharide and disaccharide concentration;
the starting liquid of step (b1) comprises a portion of the initial fruit or vegetable juice;
step (b2) comprises contacting the initial fruit or vegetable juice with the enzymatically active composite system in the bioreactor, thereby inducing an enzymatic reaction, which catalyzes a transformation of monosaccharides, disaccharides or both into at least one of at least one sugar alcohol; at least one a sugar acid; at least one oligosaccharide; at least one polysaccharide; or any combination thereof; to produce a processed juice, which has a final total monosaccharide and disaccharide concentration; and
wherein the initial total monosaccharide and disaccharide concentration is higher than the final total monosaccharide and disaccharide concentration.

63. The method of claim 62, wherein the one or more times of the enzymatic reaction according to (b) after the disinfection of (c) comprises a final repeat, wherein the time from an initial performance of the enzymatic reaction according to (b) to the final repeat is at least 30 days, during which the enzymatically active composite system is maintained within the reactor, and wherein a total microbial count of the processed juice produced in step (b) of the final repeat is no more than 1000 CFU/ml.

64. The method of claim 62, wherein:
the aqueous disinfecting composition is a food grade composition;
the support comprises a combination of carrageenan and xanthan gum;
the enzymatically active agent comprises a plurality of non-proliferating bacterial cells, comprising glucose fructose oxidase reductase (GFOR), glucose oxidase (GOX) or sucrose fructosyltransferase (SFTase);
the final total monosaccharide and disaccharide concentration is at least 25% lower than the initial total monosaccharide and disaccharide concentration;
the initial juice has an initial total oligosaccharide and polysaccharide concentration and the processed juice has a final total oligosaccharide and polysaccharide concentration, which is at least 50% higher that the initial total oligosaccharide and polysaccharide concentration;
the one or more times of the enzymatic reaction according to (b) after the disinfection of (c) comprises a final repeat, wherein the time from an initial performance of the enzymatic reaction according to (b) to the final repeat is at least 50 days, during which the enzymatically active composite system is maintained within the reactor, and wherein a total microbial count of the processed juice produced in step (b) of the final repeat is no more than 100 CFU/ml;
said fruit or vegetable juice is selected from a group consisting of orange juice, grape juice, apple juice, pear juice, cherry juice, strawberry juice, and pineapple juice.
